(19) United States
(12) Patent Application Publication
(10) Pub. No.: US 2005/0154022 A1
(43) Pub. Date: Jul. 14, 2005

(54) 4-aryl piperidines

(75) Inventors: Mohammad R. Marzabadi, Ridgewood, NJ (US); John M. Wetzel, Fairlawn, NJ (US); Chien-An Chen, Flushing, NY (US); John E. DeLeon, North Bergen, NJ (US); Yu Jiang, Jersey City, NJ (US); Kai Lu, Lake Hiawatha, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S

(21) Appl. No.: 11/034,611

(22) Filed: Jan. 13, 2005

Related U.S. Application Data
(60) Provisional application No. 60/536,585, filed on Jan. 14, 2004.

(51) Int. Cl. A61K 31/4545; C07D 41/14

(52) U.S. Cl. 514/318; 546/194

(57) ABSTRACT

This invention is directed to 4-aryl piperidines and related heterocyclic compounds which are selective antagonists for melanin concentrating hormone-1 (MCH1) receptors. The invention provides a pharmaceutical composition comprising a therapeutically effective amount of the compound of the invention and a pharmaceutically acceptable carrier. This invention provides a pharmaceutical composition made by combining a therapeutically effective amount of the compound of this invention and a pharmaceutically acceptable carrier. This invention further provides a process for making a pharmaceutical composition comprising combining a therapeutically effective amount of the compound of the invention and a pharmaceutically acceptable carrier. This invention also provides a method of reducing the body mass of a subject which comprises administering to the subject an amount of a compound of the invention effective to reduce the body mass of the subject. This invention further provides a method of treating a subject suffering from depression and/or anxiety which comprises administering to the subject an amount of a compound of the invention effective to treat the subject's depression and/or anxiety.

This application claims the benefit of U.S. Provisional Application No. 60/536,585, filed on Jan. 14, 2004, the contents of which is hereby incorporated by reference.

### FIELD OF THE INVENTION

The instant invention is concerned with 4-aryl-piperidines and related heterocyclic compounds as therapeutic agents for the treatment of physiological ailments such as certain psychiatric conditions including, but not limited to, depression and anxiety. Additionally, the therapeutic agent of the instant invention may be used to treat obesity or urge incontinence.

### BACKGROUND OF THE INVENTION

Melanin-concentrating hormone (MCH) is a cyclic neuropeptide originally isolated from salmonid (teleost fish) pituitaries (Kawauchi et al., 1983). The identification of a G-protein coupled receptor for MCH was published (Chambers et al., 1999; Saito et al., 1999). These groups identified MCH as the endogenous ligand for the human orphan G-protein coupled receptor SLC-1 (Lakaye et al., 1998). Since this discovery, it was discovered that mammalian MCH (19 amino acids) is highly conserved between rat, mouse, and human, exhibiting 100% amino acid identity. The rat homologue of this receptor, now called MCH1, was reported to be localized in regions of the rat brain.

In our own studies, MCH1 antagonists have been evaluated in several animal models that are well known as predictive for the efficacy of compounds in humans, see Borowsky, B., et al. (2002). These experiments suggest that MCH1 antagonists may be useful to treat depression and/or anxiety. After mapping the binding sites for [(3) H] SNAP-7941, a selective and potent MCH1 antagonist in rat brain, we evaluated its effects in a series of behavioral models. SNAP-7941 produced effects similar to clinically used antidepressants and anxiolytics in three animal models of depression/anxiety: the rat forced-swim test, rat social interaction and guinea pig maternal-separation vocalization tests. These observations suggest that an MCH1 antagonist may be used to treat depression and/or anxiety.

Additionally, the link between MCH1 and the effects of MCH on feeding has been suggested by recent reports on the phenotype of MCH-1 knockout mice. Two groups have independently shown that the targeted disruption of the MCH-1 receptor gene (MCH1 knockout) in mice results in animals that are hyperphagic but are lean and have decreased body mass relative to wild-type littermates (Marsh et al, 2002; Chen et al, 2002). The decrease in body mass is attributed to an increase in metabolism. Each group demonstrated that the MCH-1 knockout mice are resistant to diet-induced obesity, and generally exhibit weights similar to littermates maintained on regular chow.

Synthetic antagonist molecules for the MCH-1 receptor have been described in the literature. Bednarek et al. (2002) have reported on the synthesis of high affinity peptide antagonists of MCH-1. A small molecule antagonist of MCH1 has been described by Takekawa et al. (2002).

In our laboratories, we have discovered small molecules that are antagonists of the MCH1 receptor. Accordingly, the compounds of the instant invention may be used to treat the indications listed above.

### SUMMARY OF THE INVENTION

Accordingly, the present invention relates to compounds having the structure: 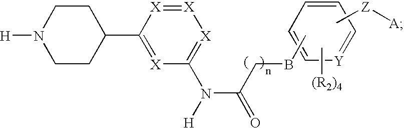
wherein each X is independently CR₁ or N, with the proviso that if one X is N then the remaining X are CR₁;
wherein each R₁ is independently —H, —F, —Cl, —Br, —I, —CN, —NO₂, straight chained or branched C₁-C₇ alkyl, alkyloxy, monofluoroalkyl or polyfluoroalkyl, or C₃-C₆ cycloalkyl-C₁-C₇ alkyl;
wherein each R₂ is independently —H, —F, —Cl, —Br, —I, —CN, —NO₂ or straight chained or branched C₁-C₇ alkyl, monofluoroalkyl or polyfluoroalkyl;
wherein n is an integer from 2 to 6 inclusive;
wherein B is CH₂, CHOH, O or CO;
wherein Y is C or N;
wherein Z is O, S, SO, SO₂, CH₂, CO, CHOH or null; and wherein A is phenyl or heteroaryl, where the phenyl or heteroaryl is optionally substituted with three or less R₂;
or a pharmaceutically acceptable salt thereof.

In one embodiment of the invention, the compound is selected from one of the specific compounds disclosed in the Detailed Description of the Invention.

In an embodiment of the present invention the compound is enantiomercially pure. In another embodiment of the invention, the compound is diastereomerically pure. In a further embodiment, the compound is enantiomerically and diasteromerically pure.

The present invention further provides a pharmaceutical composition that comprises a therapeutically effective amount of a compound of the present invention and a pharmaceutically acceptable carrier.

The present invention further provides a pharmaceutical composition made by admixing a compound of the present invention and a pharmaceutically acceptable carrier.

The present invention also provides a process for making a pharmaceutical composition comprising admixing a compound of the present invention and a pharmaceutically acceptable carrier.

The invention further provides a method of treating a subject suffering from an affective disorder selected from the group consisting of depression, major depression, bipolar disorder, agoraphobia, specific phobia, social phobia, obsessive-compulsive disorder, post-traumatic stress disorder, acute stress disorder and anxiety comprising administering to the subject a therapeutically effective amount of the compound of the invention. In a separate embodiment of the invention, the disorder is depression or anxiety.

Additionally, the invention further provides a method of treating a subject suffering from a urinary disorder selected from the group consisting of urinary incontinence, urge incontinence, urinary frequency, urinary urgency, nocturia and enuresis comprising administering to the subject a therapeutically effective amount of the compound of the invention. In a separate embodiment of the invention, the disorder is urge incontinence.

The invention further provides a method of treating a subject suffering from an eating disorder selected from the group consisting of obesity, bulimia, bulimia nervosa and anorexia nervosa comprising administering to the subject a therapeutically effective amount of the compound of the invention. In a separate embodiment of the invention, the disorder is obesity.

### DETAILED DESCRIPTION OF THE INVENTION

In the present invention, the term straight chained or branched C₁-C₇ alkyl refers to a saturated hydrocarbon having from one to seven carbon atoms inclusive. Examples of such substituents include, but are not limited to, methyl, ethyl, 1-propyl, 2-propyl, 1-butyl, 2-butyl, 2-methly-2-propyl and 2-methly-1-propyl. The term straight chained or branched C₁-C₇ alkyloxy refers to a saturated alkoxy group having from one to seven carbon atoms inclusive with the open valency on the oxygen. Examples of such substituents include, but are not limited to, methoxy, ethoxy, n-butoxy, etc. The term, C₃-C₆ cycloalkyl-C₁-C₇ alkyl designates a saturated alkyl hydrocarbon substituted with a monocyclic carbocycle ring having three to seven carbon atoms attached via the C₁-C₇ alkyl moiety. Examples of such substituents include, but are not limited to, cyclopropyl-methyl, cyclopentyl-ethyl, cyclohexyl-n-propyl, etc.

As used in the present invention, the term “heteroaryl” is used to include five and six membered unsaturated rings that contain one or more oxygen, sulfur, or nitrogen atoms. Examples of heteroaryl groups include, but are not limited to, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridinyl, pyridazinyl, pyrimidinyl, pyrazinyl, and triazinyl.

The invention provides for each pure stereoisomer of any of the compounds described herein. Such stereoisomers may include enantiomers, diastereomers, or E or Z alkene or imine isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3 B 5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition the compounds of the present invention may be present as a mixture of enantiomers, diastereomers or isomers. Furthermore, two or more of the compounds may be present to form a racemic or diastereomeric mixture. The compounds of the present invention are preferably 80% pure, more preferably 90% pure, and most preferably 95% pure. Included in this invention are pharmaceutically acceptable salts and complexes of all of the compounds described herein. The acids and bases from which these salts are prepared include, but are not limited to, the acids and bases listed herein. The acids include, but are not limited to, the following inorganic acids: hydrochloric acid, hydrobromic acid, hydroiodic acid, sulfuric acid and boric acid. The acids include, but are not limited to, the following organic acids: acetic acid, malonic acid, succinic acid, fumaric acid, tartaric acid, maleic acid, citric acid, methanesulfonic acid, benzoic acid, glycolic acid, lactic acid and mandelic acid. The bases include, but are not limited to ammonia, methylamine, ethylamine, propylamine, dimethylamine, diethylamine, trimethylamine, triethylamine, ethylenediamine, hydroxyethylamine, morpholine, piperazine and guanidine. This invention further provides for the hydrates and polymorphs of all of the compounds described herein.

The present invention includes within its scope prodrugs of the compounds of the invention. In general, such prodrugs will be functional derivatives of the compounds of the invention which are readily convertible in vivo into the required compound. Thus, in the present invention, the term “administering” shall encompass the treatment of the various conditions described with a compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985. The present invention further includes metabolites of the compounds of the present invention. Metabolites include active species produced upon introduction of compounds of this invention into the biological milieu.

As referred to in the Summary of the Invention, this invention provides a pharmaceutical composition comprising a therapeutically effective amount of the compound of the invention and a pharmaceutically acceptable carrier. In one embodiment, the amount of the compound is from about 0.01 mg to about 800 mg. In another embodiment, the amount of the compound is from about 0.01 mg to about 500 mg. In yet another embodiment, the amount of the compound is from about 0.1 mg to about 250 mg. In another embodiment, the amount of the compound is from about 0.1 mg to about 60 mg. In yet another embodiment, the amount of the compound is from about 1 mg to about 20 mg. In a further embodiment, the carrier is a liquid and the composition is a solution. In another embodiment, the carrier is a solid and the composition is a tablet. In another embodiment, the carrier is a gel and the composition is a capsule, suppository or a cream. In a further embodiment the compound may be formulated as a part of a pharmaceutically acceptable transdermal patch. In yet a further embodiment, the compound may be delivered to the subject by means of a spray or inhalant. This invention also provides a pharmaceutical composition made by admixing a therapeutically effective amount of the compound of this invention and a pharmaceutically acceptable carrier. This invention provides a process for making a pharmaceutical composition comprising admixing a therapeutically effective amount of the compound of this invention and a pharmaceutically acceptable carrier.

A solid carrier can include one or more substances which may also act as endogenous carriers (e.g. nutrient or micronutrient carriers), flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, coloring agents, viscosity regulators, stabilizers or osmoregulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate or isopropyl myristate. Sterile liquid carriers are useful in sterile liquid form compositions for parenteral administration. The liquid carrier for pressurized compositions can be a halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings. The compound can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The compound can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions. Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration. In the subject application a “therapeutically effective amount” is any amount of a compound which, when administered to a subject suffering from a disease against which the compounds are effective, causes reduction, remission, or regression of the disease. In a subject application, a “subject” is a vertebrate, a mammal or a human.

The present invention provides a method of treating overactive bladder with symptoms of urge urinary incontinence, urgency and/or frequency in a subject, which comprises administering to the subject an amount of a compound of the invention effective to treat the subject's overactive bladder. This invention also provides a method of alleviating urge urinary incontinence in a subject suffering from overactive bladder, which comprises administering to the subject an amount of a compound of the invention effective to alleviate the subject's urge urinary incontinence. This invention further provides a method of alleviating urinary urgency in a subject suffering from overactive bladder, which comprises administering to the subject an amount of a compound of the invention effective to alleviate the subject's urinary urgency. Additionally, this invention provides a method of alleviating urinary frequency in a subject suffering from overactive bladder, which comprises administering to the subject an amount of a compound of the invention effective to alleviate the subject's urinary frequency. The present invention also provides a method of treating a subject suffering from a urinary disorder, which comprises administering to the subject an amount of a compound of the invention effective to treat the subject's urinary disorder. In some embodiments the urinary disorder is urinary incontinence, overactive bladder, urge incontinence, urinary frequency, urinary urgency, nocturia or enuresis. Overactive bladder and urinary urgency may or may not be associated with benign prostatic hyperplasia. The present invention provides a method of alleviating the symptoms of a disorder which is susceptible to treatment by antagonism by the MCH1 receptor, in a subject, which comprises administering to the subject an amount of an MCH1 antagonist effective to alleviate the symptoms, wherein the MCH1 antagonist is anyone of the compounds of the invention.

In an embodiment of the invention, the subject is a vertebrate, a mammal, a human or a canine. In another embodiment, the compound is administered orally. In yet another embodiment, the compound is administered in combination with food. This invention provides a method of modifying the feeding behavior of a subject which comprises administering to the subject an amount of a compound of the invention effective to decrease the consumption of food by the subject. This invention also provides a method of treating an eating disorder in a subject which comprises administering to the subject an amount of a compound of this invention effective to decrease the consumption of food by the subject. In an embodiment of the present invention, the eating disorder is bulimia, obesity or bulimia nervosa. In an embodiment of the present invention, the subject is a vertebrate, a mammal, a human or a canine. In a further embodiment, the compound is administered in combination with food. The present invention further provides a method of reducing the body mass of a subject which comprises administering to the subject an amount of a compound of the invention effective to reduce the body mass of the subject.

The present invention also provides a method of treating a subject suffering from major depressive disorder, dysthymic disorder, bipolar I and II disorders, schizoaffective disorder, cognitive disorders with depressed mood, personality disorders, insomnia, hypersomnia, narcolepsy, circadian rhythm sleep disorder, nightmare disorder, sleep terror disorder, sleepwalking disorder, obsessive-compulsive disorder, panic disorder, with or without agoraphobia, posttraumatic stress disorder, social anxiety disorder, social phobia and generalized anxiety disorder. The present invention also provides a method of treating a subject suffering from depression which comprises administering to the subject an amount of a compound of this invention effective to treat the subject's depression. The present invention further provides a method of treating a subject suffering from anxiety which comprises administering to the subject an amount of a compound of this invention effective to treat the subject's anxiety. The present invention also provides a method of treating a subject suffering from depression and anxiety which comprises administering to the subject an amount of a compound of this invention effective to treat the subject's depression and anxiety.

Additionally, the invention provides certain embodiments of the present invention. In one embodiment, n is an integer from 2 to 5 inclusive. In another embodiment, n is 2 or 3.

In one embodiment, each R₁ is independently —H, —F, —Cl, straight chained or branched C₁-C₄ alkyl or alkoxy, or C₃-C₆ cycloalkyl-C₁-C₄ alkyl. In one embodiment, each R₂ is independently —H, —F, —Cl, or straight chained or branched C₁-C₄ alkyl, monofluoroalkyl or polyfluoroalkyl.

In one embodiment, one X is N.

In one embodiment, each X is C.

In one embodiment, A is pyridinyl optionally substituted with three or less R₂.

In one embodiment, A is thienyl optionally substituted with three or less R₂.

In one embodiment, A is furanyl optionally substituted with three or less R₂.

In one embodiment, A is thiazolyl optionally substituted with three or less R₂.

In one embodiment, A is imidazolyl optionally substituted with three or less R₂.

In one embodiment, A is pyrazolyl optionally substituted with three or less R₂.

In one embodiment, A is oxazolyl optionally substituted with three or less R₂.

In one embodiment, A is triazinyl optionally substituted with three or less R₂.

In one embodiment, A is phenyl optionally substituted with three or less R₂.

In one embodiment, Z is S, SO or SO₂.

In one embodiment, Z is CH₂, CO or CHOH.

In one embodiment, Z is O or null.

In one embodiment, B is CH₂.

In one embodiment, Z is O.

In one embodiment, Z is null.

In one embodiment, is CO.

In one embodiment, is null.

The invention will be better understood from the Experimental Details which follow. However, one skilled in the art will readily appreciate that the specific methods and results discussed therein are merely illustrative of the invention as described more fully in the claims which follow thereafter.

### I. SYNTHETIC SCHEMES

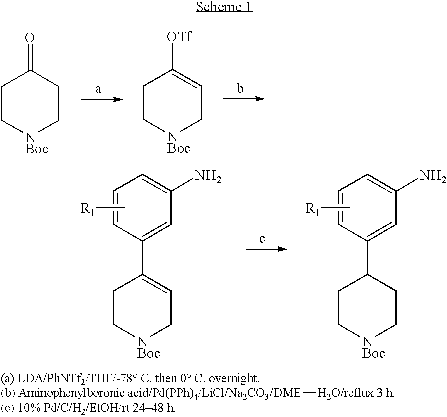

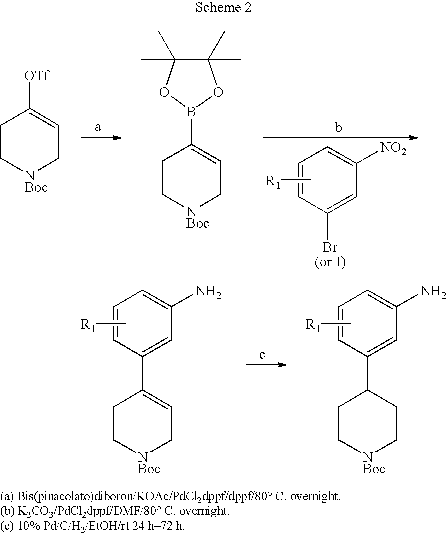

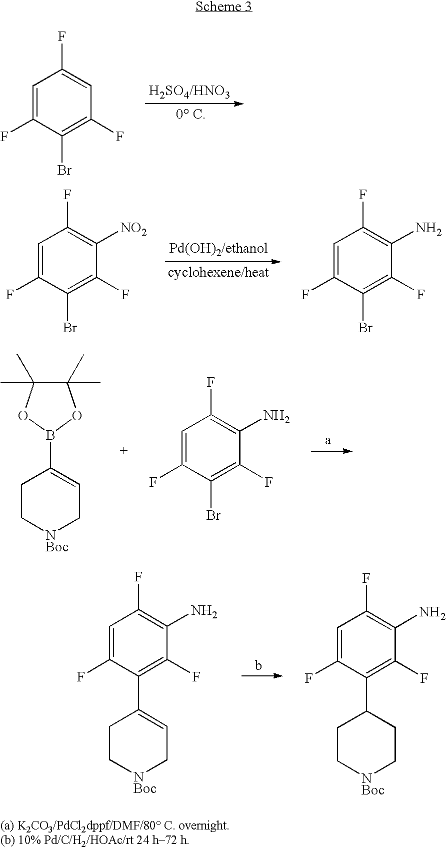

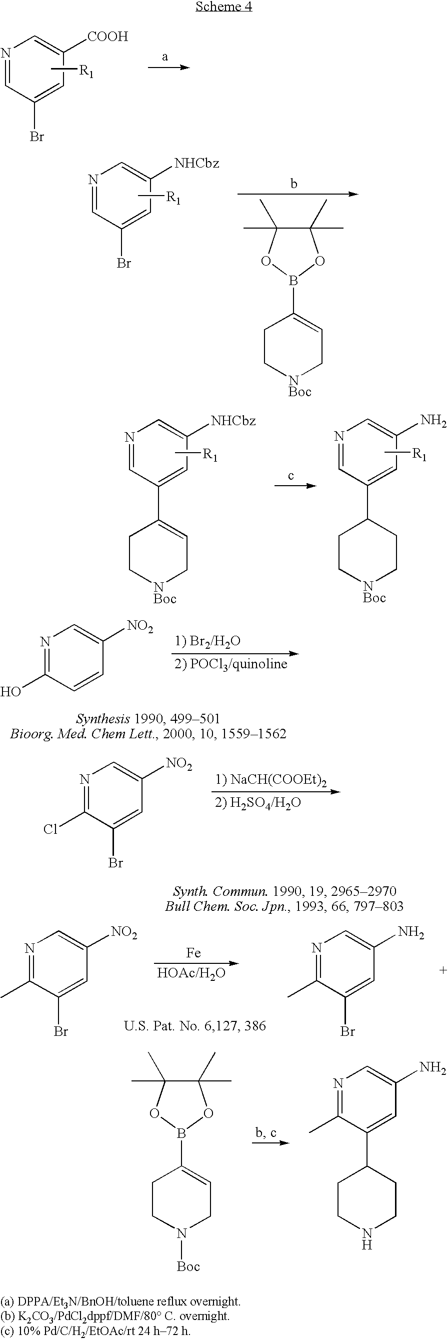

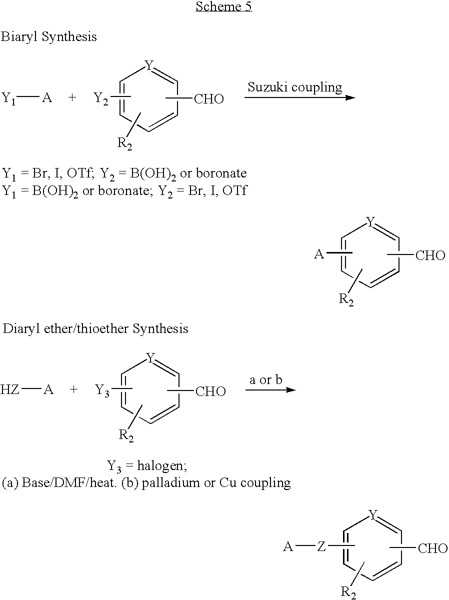

For biphenyl, diaryl ether and diaryl thioether syntheses, the starting materials are available from commercial sources or alternatively may be prepared from a variety of intermediates known to those skilled in the art. Further information can be found in the following references: Suzuki, 1995, Chem. Rev. 95, 2457; Suzuki, 1999, J. Organomet. Chem. 576(1-2), 147-168; Schopfer, 2001, Tetrahedron, 57, 3069-3073; Venkataraman, 2002, Org. Lett. 16, 2803-2806; Hartwig, 1998, Angew. Chem. Int. Ed. 37, 2046-2067 and the references cited therein.

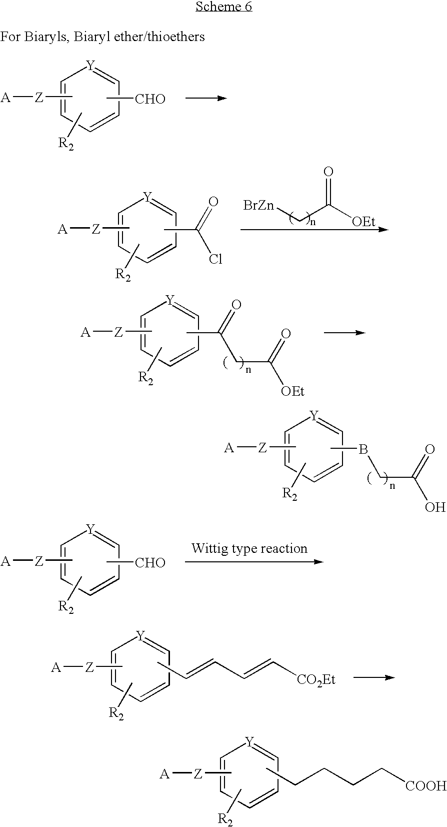

For organozinc reagents, the starting materials are available from commercial sources or alternatively may be prepared from a variety of intermediates known to those skilled in the art. Further information can be found in the following references: Rieke, 1991, J. Org. Chem. 56, 1445; Rieke, 1997, Tetrahedron 53, 1925 and the references cited therein). For wittig type reaction, the starting materials are available from the commercial sources or alternatively may be prepared from a variety of intermediates known to those skilled in the art. Further information can be found in the following references: Fesik, 1997, J. Med. Chem. 40, 3144-3150 and the references cited therein.

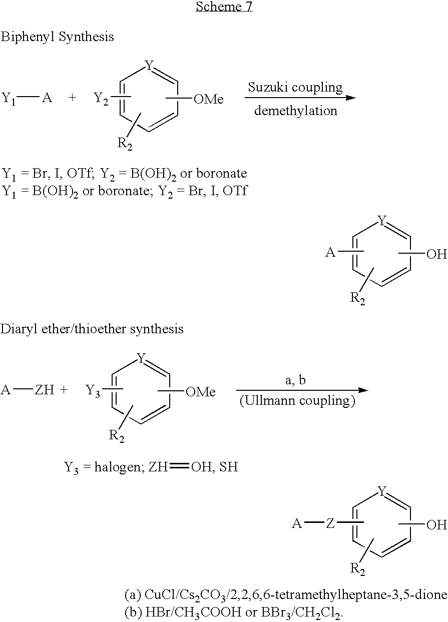

For biphenyl, diaryl ether and diaryl thioether syntheses, the starting materials are available from the commercial sources or alternatively may be prepared from a variety of intermediates known to those skilled in the art. Further information can be found in the following references: Suzuki, 1995, Chem. Rev. 95, 2457; Suzuki, 1999, J. Organomet. Chem. 576(1-2), 147-168; Schopfer, 2001, Tetrahedron, 57, 3069-3073; Venkataraman, 2002, Org. Lett. 16, 2803-2806; Hartwig, 1998, Angew. Chem. Int. Ed. 37, 2046-2067 and the references cited therein.

For Ullmann coupling syntheses, the starting materials are available from commercial sources or alternatively may be prepared from a variety of intermediates known to those skilled in the art. Further information can be found in the following references: Song, 2002, Org. Lett. 4, 1623-1626 and the references cited therein.

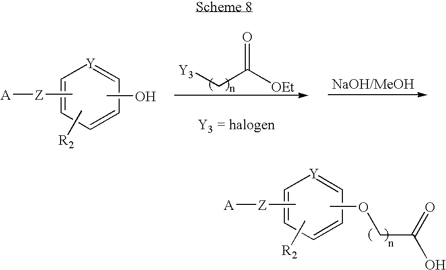

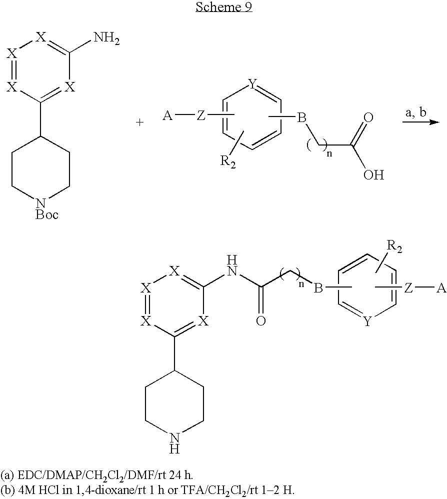

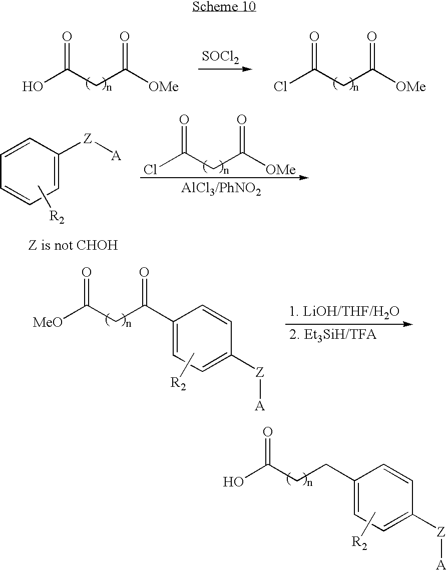

The biaryl intermediates, used in scheme 9, may alternatively be synthesized via a Friedel-Crafts route using activated carboxylic acids and biaryl systems as depicted in Scheme 10.

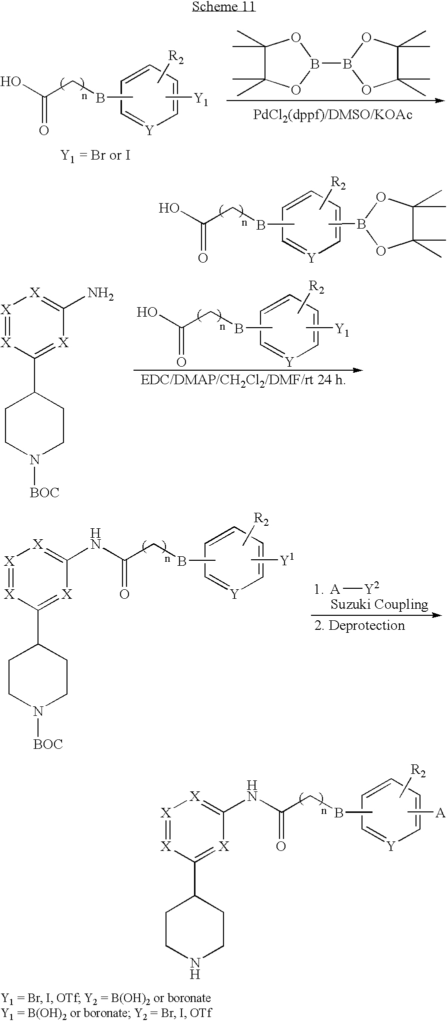

The biaryls depicted in Scheme 11 may be synthesized via a Suzuki route using boronic acids/boronates and aryl halides/triflates as described in Scheme 11.

### II. DETAILED SYNTHESIS OF EXAMPLES

The following examples are for the purpose of illustrating methods useful for making compounds of this invention.

General Methods: All reactions were performed under a nitrogen atmosphere and the reagents, neat or in appropriate solvents, were transferred to the reaction vessel via syringe and cannula techniques. Anhydrous solvents were purchased from the Aldrich Chemical Company and used as received. The examples described in the patent were named using the ACD/Name Program (version 4.01, Advanced Chemistry Development Inc., Toronto, Ontario, M5H2L3, Canada). The ¹H NMR and ¹³C NMR spectra were recorded at 300 and 75 MHz, respectively (GE QE Plus) or at 400 and 100 MHz, respectively (Bruker Avance) in CDCl₃ as solvent with tetramethylsilane as the internal standard unless otherwise noted. Chemical shifts (δ) are expressed in ppm, coupling constants (J) are expressed in Hz, and splitting patterns are described as follows: s=singlet; d=doublet; t=triplet; q=quartet; quintet; sextet; septet; br=broad; m=mutiplet; dd=doublet of doublets; ddd=double of doublets of doublets; dt=doublet of triplets; td=triplet of doublets; dm=doublet of multiplets. Elemental analyses were performed by Robertson Microlit Laboratories, Inc. Unless otherwise noted, mass spectra were obtained using electrospray ionization (ESMS, Micromass Platform II or Quattro Micro) and (M+H)⁺ or (M−H)⁻ is reported. Thin-layer chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Flash column chromatography was performed on Merck silica gel 60 (230-400 mesh). Melting points (mp) were determined in open capillary tubes on a Mel-Temp apparatus and are uncorrected. Microwave experiments were carried out using a Biotage Emyrs Optimizer™ or Smithcreator. Bond elute cartridge: Isolute silica solid phase extraction cartridge with silanol groups on the surface of the silica particle is supplied by Argonaut Technologies.

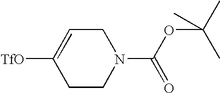

TERT-BUTYL 4-{[(TRIFLUOROMETHYL)SULFONYL]OXY}-3,6-DIHYDRO-1(2H)-PYRIDINE CARBOXYLATE: n-Butyl lithium (17.6 mL, 44.2 mmol, 2.5 M in hexanes) was added to a solution of diisopropylamine (96.2 mL, 44.2 mmol) in anhydrous THF (40.0 mL) at 0° C. and the resulting mixture was stirred for 20 minutes. The reaction mixture was cooled to −78° C. and tert-butyl 4-oxo-1-piperidinecarboxylate (Aldrich Chemical Company, 7.97 g, 40.0 mmol) in THF (40.0 mL) was added dropwise to the reaction mixture, which was then stirred for 30 minutes. Tf₂NPh (42.0 mmol, 15.0 g) in THF (40.0 mL) was added dropwise to the reaction mixture and the reaction mixture was stirred at 0° C. overnight. The reaction mixture was concentrated in vacuo, redissolved in hexanes:EtOAc (9:1), passed through a plug of alumina and the alumina plug was washed with hexanes:EtOAc (9:1). The combined extracts were concentrated in vacuo to yield the desired product (16.5 g) which was contaminated with some starting material Tf₂NPh: ¹H NMR (400 MHz, CDCl₃) δ 5.77 (s, 1H), 4.05 (dm, 2H, J=3.0 Hz), 3.63 (t, 2H, J=5.7 Hz), 2.45 (m, 2H), 1.47 (s, 9H).

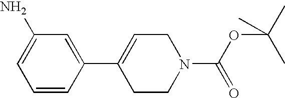

TERT-BUTYL 4-(3-AMINOPHENYL)-3,6-DIHYDRO-1(2H)-PYRIDINE CARBOXYLATE: A degassed mixture of 2.0 M aqueous Na₂CO₃ solution (4.20 mL), tert-butyl 4-{[(trifluoromethyl)sulfonyl]oxy}-3,6-dihydro-1(2H)-pyridine carboxylate (0.500 g, 1.51 mmol), 3-aminophenylboronic acid hemisulfate (0.393 g, 2.11 mmol), lithium chloride (0.191 g, 4.50 mmol) and tetrakis-triphenylphosphine palladium (0.080 g, 0.075 mmol) in dimethoxyethane (5.00 mL) was heated at reflux temperature for 3 hours under Argon. The organic layer of the cooled reaction mixture was separated and the aqueous layer was washed with ethyl acetate (3×50 mL). The combined organic solutions were dried and concentrated in vacuo. The crude product was chromatographed (silica, hexanes:EtOAc:dichloromethane 6:1:1 with 1% isopropylamine) to give the desired product (0.330 g, 81%). ¹H NMR (400 MHz, CDCl₃) δ 7.12 (t, 1H, J=7.60 Hz), 6.78 (d, 1H, J=8.4 Hz), 6.69 (t, 1H, J=2.0 Hz), 6.59 (dd, 1H, J=2.2, 8.0 Hz), 6.01 (br, 1H), 4.10-4.01 (d, 2H, J=2.4 Hz) 3.61 (t, 2H, J=5.6 Hz), 2.52-2.46 (m, 2H), 1.49 (s, 9H); ESMS m/e: 275.2 (M+H)⁺. Anal. Calc. for C₁₆H₂₄N₂O₂: C, 70.04; H, 8.08; N, 10.21. Found: C, 69.78; H, 7.80; N, 9.92.

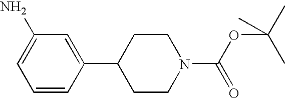

TERT-BUTYL 4-[3-(AMINO)PHENYL]-1-PIPERIDINECARBOXYLATE: A mixture of tert-butyl 4-(3-aminophenyl)-3,6-dihydro-1(2H)-pyridinecarboxylate (3.10 g, 11.3 mmol) and 10% Pd/C (1.00 g) in ethanol (100 mL) was hydrogenated at room temperature using the balloon method for 2 days. The reaction mixture was filtered through Celite and washed with ethanol. The combined ethanol extracts were concentrated in vacuo and the residue was chromatographed on silica (dichloromethane: methanol:isopropylamine 95:5:1) to give the desired product (2.63 g, 84%). ¹H NMR (400 MHz, CDCl₃) δ 7.10 (t, 1H, J=7.6 Hz), 6.62 (d, 1H, J=8.4 Hz), 6.60-6.59 (m, 2H), 4.27-4.18 (m, 2H), 3.62-3.58 (m, 2H), 2.80-2.72 (m, 2H), 2.62-2.59 (m, 1H), 1.89-1.52 (m, 4H), 1.49 (s, 9H); ESMS m/e: 277.2 (M+H)⁺.

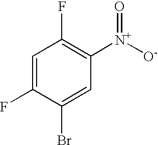

1-BROMO-2,4-DIFLUORO-5-NITROBENZENE: To a 0° C. mixture of 1-bromo-2,4-difluorobenzene (20.0 g; 11.7 mL; 0.100 mol) and H₂SO₄ (76.8 mL) was added HNO₃ (68.0 mL) over 45 min at such a rate that the internal temperature was <7° C. The resulting mixture was stirred for 1 h at 0° C, poured into ice water (400 mL), stirred vigorously for 2-3 min and extracted with CH₂Cl₂ (400 mL). The CH₂Cl₂ extract was washed with brine (1×500 mL), dried over Na₂SO₄, filtered and evaporated to give the product as a yellow oil (23.5 g, 95%). ¹H NMR (300 MHz, CDCl₃) δ 7.14 (ddd, J=0.3, 7.8, 9.9 Hz, 1H), 8.39 (t, J=7.2 Hz, 1H).

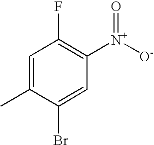

2-BROMO-5-FLUORO-4-NITRO TOLUENE: To a refluxing mixture of nitronium tetrafluoroborate (11.6 g; 87.0 mmol) and CH₂Cl₂ (60.0 mL) was added 2-bromo-5-fluoro toluene (15.0 g, 10.0 mL, 79.0 mmol) over 5 minutes. The mixture was stirred at reflux for 4.5 h, cooled and poured into ice water (150 mL). The aqueous portion was extracted with CH₂Cl₂ (1×150 mL). The combined CH₂Cl₂ extracts were washed with brine (100 mL), dried over Na₂SO₄, filtered and evaporated to give 18.3 g of a crude product that was treated with hexane and evaporated until the appearance of crystals. The mixture was cooled to −70° C. and the hexane was decanted away from the resulting solid. Residual hexane was removed by evaporation to give 9.77 g of the product as a semi-solid (53%). The mother liquors were evaporated and purified by column chromatography (silica gel, 2% EtOAc in hexane). Evaporation of the appropriate fractions gave 1.0 g of the product (59% in total). ¹H NMR (300 MHz, CDCl₃) δ 2.48 (s, 3H), 7.20 (d, J=11.7 Hz, 1H), 8.26 (d, J=6.9 Hz, 1H).

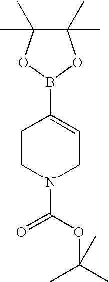

TERT-BUTYL 4-(4,4,5,5-TETRAMETHYL-1,3,2-DIOXABOROLAN-2-YL)-3,6-DIHYDRO-1(2H)-PYRIDINECARBOXYLATE: To a 50-mL RB-flask, charged with bis(pinacolato)diboron (422 mg, 1.66 mmol), KOAc (444 mg, 4.53 mmol), PdCl₂dppf (37.0 mg, 3.00 mol %) and dppf (25.0 mg, 3.00 mol %) was added a solution of tert-butyl 4-{[(trifluoromethyl)sulfonyl]oxy}-3,6-dihydro-1(2H)-pyridinecarboxylate (500 mg, 1.51 mmol) in 1,4-dioxane (10.0 mL) at room temperature under argon. The mixture was heated at 80° C. overnight. After cooling to room temperature, the mixture was filtered through Celite and the Celite was washed with EtOAc (3×20 mL). The filtrates were concentrated in vacuo. The resulting residue was dissolved in EtOAc and washed with H₂O and brine, dried over MgSO₄, filtered and concentrated in vacuo. The crude product was purified by flash chromatography (1:9 EtOAc:hexane) to give tert-butyl 4-(4,4,5,5-tetramethyl-1,3,2-dioxaborolan-2-yl)-3,6-dihydro-1(2H)-pyridinecarboxylate (355 mg, 76%): ¹H NMR (400 MHz, CDCl₃) δ 6.60-6.34 (br, 1H), 4.06-3.86 (br, 2H), 3.55-3.34 (br, 2H), 2.35-2.09 (br, 2H), 1.46 (s, 9H), 1.26 (s, 12H); ESMS m/e: 310.4 (M+H)⁺.

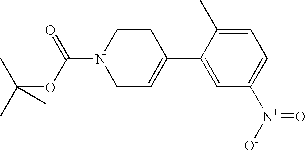

TERT-BUTYL 4-(5-NITRO-2-METHYLPHENYL)-3,6-DIHYDRO-1(2H)-PYRIDINE CARBOXYLATE: To a solution of 2-bromo-1-methyl-4-nitrobenzene (14.0 g, 64.8 mmol) and DMF (400 mL) was added tert-butyl 4-(4,4,5,5-tetramethyl-1,3,2-dioxaborolan-2-yl)-3,6-dihydro-1(2H)-pyridinecarboxylate (20.0 g; 64.8 mmol), K₂CO₃ (27.0 g; 194 mmol) and PdCl₂dppf CH₂Cl₂ (3.20 g; 3.90 mmol; 6 mol % catalyst loading). The resulting mixture was heated at 80° C. under nitrogen for 6 h, cooled to room temperature, allowed to stand for 18 h then cooled to 4° C. Water (400 mL) was added over 10 min at such a rate that the temperature was <35° C. EtOAc (400 mL) was added, the mixture was stirred for 15 min and the EtOAc layer was removed. This extraction procedure was repeated with EtOAc (2×400 mL). The organic extracts were combined, washed with water (800 mL) and saturated aqueous NaCl (320 mL), filtered through a pad of Celite®, dried over MgSO₄, filtered and evaporated to give a dark residue which was purified by column chromatography (silica gel, 70:30 hexane/EtOAc). Evaporation of the appropriate fractions gave 22.0 g of the product as a solid, which was used in the next step. ¹H NMR (300 MHz, CDCl₃) δ 1.50 (s, 9H), 2.30-2.39 (m, 5H), 3.64 (t, J=5.7 Hz, 2H), 4.03-4.08 (m, 2H), 5.60-5.66 (m, 1H), 7.31 (d, J=8.4 Hz, 1H), 7.95 (d, J=2.7 Hz, 1H), 8.01 (dd, J=2.7, 8.4 Hz, 1H).

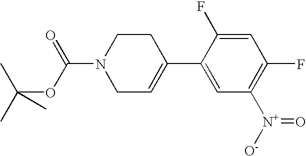

TERT-BUTYL 4-(5-NITRO-2,4-DIFLUOROPHENYL)-3,6-DIHYDRO-1(2H)-PYRIDINE CARBOXYLATE: ¹H NMR (300 MHz, CDCl₃) δ 1.50 (s, 9H), 2.44-2.52 (m, 2H), 3.63 (t, J=5.7 Hz, 2H), 4.07-4.12 (m, 2H), 6.01-6.07 (m, 1H), 7.02 (t, J=10.2 Hz, 1H), 8.05 (t, J=8.1 Hz, 1H).

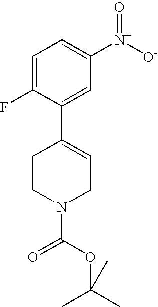

TERT-BUTYL 4-(5-NITRO-2-FLUOROPHENYL)-3,6-DIHYDRO-1(2H)-PYRIDINE CARBOXYLATE: ¹H NMR (300 MHz, CDCl₃) δ 1.49 (s, 9H), 2.48-2.55 (m, 2H), 3.64 (t, J=5.4 Hz, 2H), 4.08-4.13 (m, 2H), 6.07 (br s, 1H), 7.18 (dd, J=9.2, 9.9 Hz, 8.09-8.20 (m, 2H).

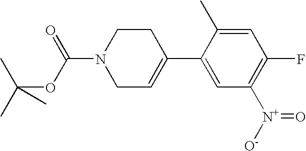

TERT-BUTYL 4-(5-NITRO-4-FLUORO-2-METHYLPHENYL)-3,6-DIHYDRO-1(2H)-PYRIDINE CARBOXYLATE: ¹H NMR (300 MHz, CDCl₃) δ 1.50 (s, 9H), 2.57-2.74 (m, 5H), 3.63 (t, J=6.3 Hz, 2H), 4.03-4.08 (m, 2H), 5.61-5.68 (m, 1H), 7.09 (d, J=11.7 Hz, 1H), 7.80 (d, J=7.8 Hz, 1H).

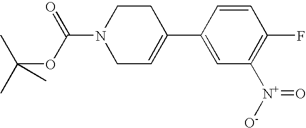

TERT-BUTYL 4-(3-NITRO-4-FLUOROPHENYL)-3,6-DIHYDRO-1(2H)-PYRIDINE CARBOXYLATE: ¹H NMR (300 MHz, CDCl₃) δ 1.50 (s, 9H), 2.48-2.56 (m, 2H), 3.63-3.69 (m, 2H), 4.08-4.14 (m, 2H), 6.10-6.16 (m, 1H), 7.22-7.30 (m, 1H (obscured by solvent)), 7.60-7.65 (m, 1H), 8.03 (dd, J=2.4, 6.9 Hz, 1H).

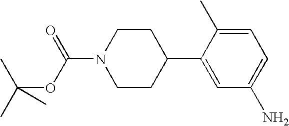

TERT-BUTYL 4-(5-AMINO-2-METHYLPHENYL)-1-PIPERIDINECARBOXYLATE: A mixture of tert-butyl 4-(5-nitro-2-methylphenyl)-3,6-dihydro-1(2H)-pyridinecarboxylate (22.0 g), ethanol (absolute, 300 mL) and 10% Pd-C (2.00 g) was held at 55-60 psi under a hydrogen atmosphere for 66 h. The mixture was filtered and concentrated to give a crude green oil (55% conversion by ¹H NMR). To the solution of the oil and ethanol (300 mL) was added 10% Pd-C (2.00 g) and the resulting mixture was held at 55-60 psi under a hydrogen atmosphere for 20 h 15 min. The mixture was filtered through Celite® and the cake was washed with ethanol (200 mL) and EtOAc (100 mL). The filtrate was concentrated, diluted with EtOAc (500 mL), dried over MgSO₄, filtered and evaporated to give 18.4 g of an oil which was dissolved in EtOAc (10 mL) and hexanes (350 mL) and allowed to stand at 5° C. for 18 h. The resulting mixture was filtered to give 13.4 g of the product as a solid (71% yield over 2 steps). ¹H NMR (300 MHz, CDCl₃) δ 1.49 (s, 9H), 1.50-1.63 (m, 2H), 1.68-1.77 (m, 2H), 2.23 (s, 3H), 2.51-2.86 (m, 3H), 3.53 (bs, 2H), 4.18-4.30 (m, 2H), 6.47 (dd, J=2.4, 8.1 Hz, 1H), 6.53 (d, J=2.4 Hz, 1H), 6.93 (d, J=8.1 Hz, 1H).

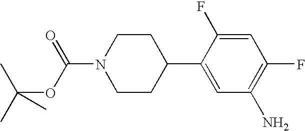

TERT-BUTYL 4-(5-AMINO-2,4-DIFLUOROPHENYL)-1-PIPERIDINECARBOXYLATE: A mixture of tert-butyl 4-(5-nitro-2,4-difluorophenyl)-3,6-dihydro-1(2H)-pyridinecarboxylate (128.0 g, 53.0 mmol), ethanol (720 mL) and 10% Pd-C (50% water by weight, 3.20 g) was held at 60 psi under a hydrogen atmosphere for 16 h. The mixture was filtered through a pad of Celite® and the Celite® pad was washed with ethanol (4×25 mL). The filtrate was concentrated to give 17.0 g of thick oil that was further dried under high vacuum for several hours. Hexanes (125 mL) were added and the mixture was cooled to 5° C. for 30 min with vigorous stirring. The resulting solid was collected by filtration and the solid cake was washed with the mother liquors then with cold hexanes (50 mL) and dried to give 15.5 g of the product as beige solid (94% yield). ¹H NMR (300 MHz, CDCl₃) δ 1.48 (s, 9H), 1.49-1.63 (m, 2H), 1.70-1.79 (m, 2H), 2.73-2.81 (m, 3H), 3.30-3.80 (br s, 2H), 4.14-4.30 (m, 2H), 6.58 (dd, J=7.5, 9.9 Hz, 1H), 6.73 (t, J=9.9 Hz, 1H); ¹⁹F NMR (282 MHz, CDCl₃) δ −134.55, −134.52, −134.48, −129.51 (t, J=8.5 Hz).

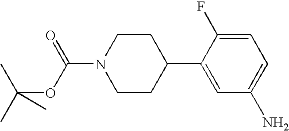

TERT-BUTYL 4-(5-AMINO-2—FLUOROPHENYL)-1-PIPERIDINECARBOXYLATE: ¹H NMR (300 MHz, CDCl₃) δ 1.48 (s, 9H), 1.50-1.66 (m, 2H), 1.73-1.82 (m, 2H), 2.73-2.98 (m, 3H), 3.51 (br s, 2H), 4.15-4.30 (m, 2H), 6.44-6.51 (m, 2H), 6.76-6.85 (m, 1H); ¹⁹F NMR (282 MHz; CDCl₃) δ −133.11, −133.09, −133.07, −133.05, −133.04.

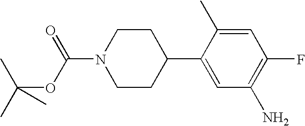

TERT-BUTYL 4-(5-AMINO-4-FLUORO-2-METHYLPHENYL)-1-PIPERIDINECARBOXYLATE: ¹H NMR (300 MHz; CDCl₃) δ 1.43-1.60 (m, 11H), 1.69 (m, 2H), 2.22 (s, 3H), 2.67-2.86 (m, 3H), 3.52-3.86 (br s, 2H), 4.16-4.32 (m, 2H), 6.60 (d, J=9.0 Hz, 1H), 6.77 (d, J=12.0 Hz, 1H); ¹⁹F NMR (282 MHz; CDCl₃) δ −139.28, −139.24, −139.23, −139.20.

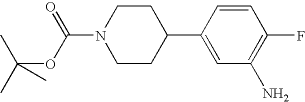

TERT-BUTYL 4-(5-AMINO-4-FLUOROPHENYL)-1-PIPERIDINECARBOXYLATE: ¹H NMR (300 MHz, CDCl₃) δ 1.49 (s, 9H), 1.50-1.62 (m, 2H), 1.72-1.81 (m, 2H), 2.45-2.58 (m, 1H), 2.70-2.83 (m, 2H), 3.68 (br s, 2H), 4.16-4.28 (m, 2H), 6.47-6.53 (m, 1H), 6.61 (dd, J=2.1, 8.7 Hz, 1H), 6.89 (dd, J=8.4, 10.8 Hz, 1H); ¹⁹F NMR (282 MHz; CDCl₃) δ −139.01 to −139.10.

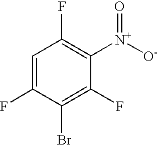

1-BROMO-3-NITRO-2,4,6,-TRIFLUOROBENZENE: To a cooled (1.3° C.) mixture of 1-bromo-2,4,6-trifluorobenzene (30.0 g; 142 mmol) and H₂SO₄ (115 mL) was added HNO₃ (68%; 102 mL) over 1 h 25 min at such a rate that the internal temperature was <8° C. The resulting mixture was stirred for 1 h 50 min at 0° C. (temperature at 1 h 50 min=4.6° C), poured onto ice (1200 mL) and water (650 mL), stirred vigorously for 30 min and extracted with CH₂Cl₂ (3×600 mL). The CH₂Cl₂ extracts were combined, washed with water (2×600 mL), dried over MgSO₄, filtered and evaporated to give the product as a clear yellow oil (35.0 g, 99% yield). ¹H NMR (300 MHz, CDCl₃) δ7.01 (ddd, J=2.4, 7.8, 9.3 Hz, 1H); ¹⁹F NMR (282 MHz; CDCl₃) δ −116.20 to −116.10, −107.73 to −107.71, −93.80 to −93.70.

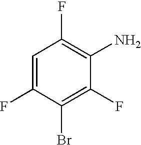

3-AMINO-1-BROMO-2,4,6-TRIFLUOROBENZENE: A mixture of 1-bromo-3-nitro-2,4,6-trifluorobenzene (15.1 g; 59.0 mmol), ethanol (590 mL), cyclohexene (177 mL) and Pd(OH)₂ (5.90 g) were heated at 80-85° C. (external temperature) under nitrogen for 2 h 5 min (Tᵢₙₜₑᵣₙₐₗ=70° C.). The mixture was cooled to 30° C., filtered through a pad of Celite® and washed with EtOAc (200 mL) and ethanol (200 mL). The yellow filtrate was concentrated and dried under high vacuum at 60° C. to give 11 g of the product as a solid (83% yield). ¹H NMR (300 MHz; CDCl₃) δ 3.60-3.80 (br s, 2H), 6.76 (ddd, J=2.4, 8.4, 10.2 Hz, 1H); ¹⁹F NMR (282 MHz, CDCl₃) δ −120.44 to −120.49, −131.20 to −131.28, −124.72 to −124.78.

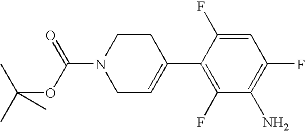

TERT-BUTYL 4-(3-AMINO-2,4,6-TRIFLUOROPHENYL)-3,6-DIHYDRO-1(2H)-PYRIDINECARBOXYLATE: ¹H NMR (300 MHz, CDCl₃) δ 1.49 (s, 9H), 2.33-2.41 (m, 2H), 3.61 (t, J=5.5 Hz, 2H), 4.03-4.07 (m, 2H), 5.74-5.81 (m, 1H), 6.63 (ddd, J=2.4, 9.6, 10.5 Hz, 1H).

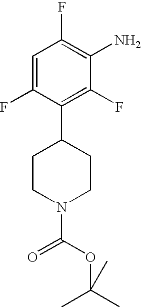

TERT-BUTYL 4-(3-AMINO-2,4,6-TRIFLUOROPHENYL)-1-PIPERIDINECARBOXYLATE: ¹H NMR (300 MHz, CDCl₃) δ 1.46 (s, 9H), 1.60-1.70 (m, 2H), 1.88-2.06 (m, 2H), 2.68-2.84 (m, 2H), 2.97-3.10 (m, 1H), 3.48-3.60 (m, 2H), 4.15-4.32 (m, 2H), 6.54-6.67 (m, 1H); ¹⁹F NMR (282 MHz; CDCl₃) δ −133.53 to −133.52, −127.48 (d, J=10.7 Hz).

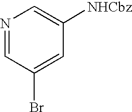

BENZYL 5-BROMO-3-PYRIDINYL CARBAMATE: To a suspension of 5-bromonicotinic acid (20.0 g, 99.0 mmol) in toluene (200 mL) was added diphenylphosphoryl azide (25.6 mL, 118.8 mmol) and Et₃N (16.6 mL, 118.8 mmol). After stirring at room temperature for 30 min, benzyl alcohol (15.4 mL, 148.5 mmol) was added. The mixture was stirred at room temperature for 1 h then refluxed overnight. After cooling to room temperature, the reaction mixture was washed with H₂O, NaHCO₃ and brine, dried over MgSO₄ and concentrated. Purification by flash chromatography (15-50% EtOAc/Hexane) provided 22.2 g (72.5 mmol, 73 %) of benzyl 5-bromo-3-pyridinylcarbamate: ¹H NMR (400 MHz, CDCl₃) δ 8.39-8.32 (m, 2 H), 8.29 (s, 1 H), 7.45-7.32 (m, 5 H), 6.94 (s, 1 H), 5.22 (s, 2 H); ESMS m/e: 307.0 (M+H)⁺.

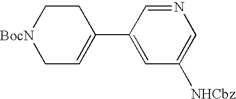

TERT-BUTYL 4-{5-[(PHENYLMETHOXY)CARBONYLAMINO]-3-PYRIDYL}-1,2,5,6-TETRAHYDROPYRIDINECARBOXYLATE: ¹H NMR (400 MHz, CDCl₃) δ 8.38-8.30 (m, 2 H), 8.10-7.97 (m, 1 H), 7.47-7.31 (m, 5 H), 7.14 (s, 1 H), 6.10 (s, 1 H), 5.22 (s, 2 H), 4.16-4.03 (m, 2 H), 3.71-3.57 (m, 2 H), 2.57-2.42 (m, 2 H), 1.49 (s, 9 H); ESMS m/e: 410.2 (M+H)⁺.

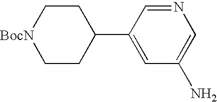

TERT-BUTYL 4-(5-AMINO-3-PYRIDINYL-1-PIPERIDINECARBOXYLATE: ¹H NMR (400 MHz, CDCl₃) δ 8.01-7.95 (m, 1 H), 7.89 (s, 1 H), 6.83 (s, 1 H), 4.39-4.09 (br, 2 H), 3.90-3.50 (br, 2 H), 2.88-2.68 (m, 2 H), 2.67-2.52 (m, 1 H), 1.88-1.71 (m, 2 H), 1.68-1.49 (m, 2 H), 1.48 (s, 9 H); ESMS m/e: 278.3 (M+H)⁺.

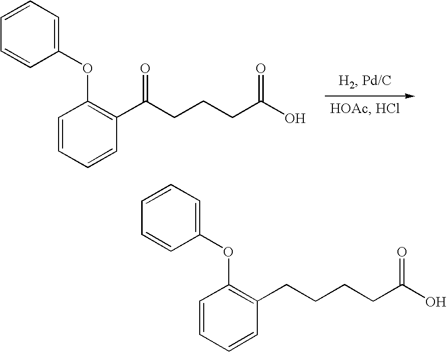

5-(2-phenoxphenyl)-valeric acid: To a solution of 5-(2-phenoxphenyl)-5-oxo-valeric acid (Rieke Metals, Inc.) (500 mg, 1.8 mmol) in acetic acid (4 mL) at room temperature was added Pd/C (10%, 50 mg) and 0.2 mL of HCl (conc.). The resulting mixture was then hydrogenated (room temperature, 100 psi, overnight). The reaction mixture was filtered through celite and the solvent was removed in vacuo. The residue was exposed to high vacuum overnight to remove the trace amounts of acetic acid. The crude product was used in the next step without any further purification (500 mg, 1.8 mmol, quantitative yield). ¹H NMR (400 MHz, CDCl₃) δ 7.35-7.21 (m, 3H), 7.19-7.12 (m, 1H), 7.11-7.01 (m, 2H), 6.96-6.83 (m, 3H), 2.65 (t, 2H, J=7.2 Hz), 2.34 (t, 2H, J=7.2 Hz), 1.73-1.59 (m, 4H); ESI-MS m/e: 269.4 (M−H)⁻.

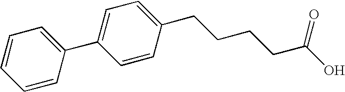

5-(4-biphenylyl)valeric acid: ¹H NMR (400 MHz, CDCl₃) δ 7.63-7.06 (m, 9H), 2.75-2.62 (m, 2H), 2.48-2.32 (m, 2H), 1.79-1.62 (m, 4H).

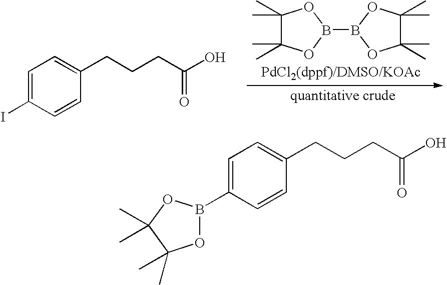

4-[4-(4,4,5,5-tetramethyl-1,3,2-dioxaborolan-2-yl)phenoxy]butanoic acid: 4-(4-Iodophenyl)butanoic acid (40.0 mg, 0.130 mmol), 4,4,5,5-tetramethyl-2-(4,4,5,5-tetramethyl(1,3,2-dioxaborolan-2-yl))-1,3,2-dioxaborolane (bis(pinocolato)diboron) (33.3 mg, 0.150 mmol), potassium acetate (54.1 mg, 0.550 mmol) and 1,1′-bis(diphenylphosphino)ferrocenedichloropalladium (5.60 mg, 5 mol %) in dimethylsulphoxide (1.50 mL) was heated at 100° C. for 18 h. The reaction mixture was concentrated in vacuo to leave a dark oil. The residue was diluted with EtOAc, filtered and the filtrate was concentrated in vacuo to leave the crude acid (38.0 mg, 95%). ESMS m/e: 291.2 (M−H)⁻.

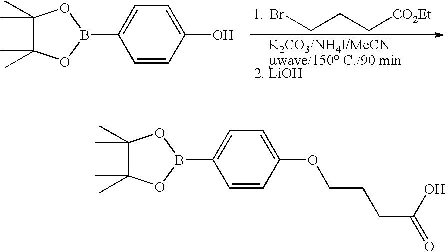

4-[4-(4,4,5,5-tetramethyl-1,3,2-dioxaborolan-2-yl)phenoxy]butanoic acid: A mixture of 4-(4,4,5,5-tetramethyl-1,3,2-dioxaborolan-2-yl)phenol, (2.00 g, 10.0 mmol), ethyl 4-bromobutanoate, (2.00 mL, 14.9 mmol), K₂CO₃ (2.40 g, 17.0 mmol), NH₄I (1.00 g, 7.00 mmol) and acetonitrile (14 mL) was heated at 140° C. for a combined total of 90 min with periodic monitoring. The reaction mixture was partitioned between water (100 mL) and diethyl ether (50 mL), separated and washed with diethyl ether (50 mL). The combined organic extracts were dried over sodium sulfate, then concentrated in vacuo to leave a yellow oil. The crude product was purified on a bond elute cartridge (70 g, performed twice) eluting with pentane followed by pentane:diethyl ether, 4:1 then 1:1 to give ethyl 4-[4-(4,4,5,5-tetramethyl-1,3,2-dioxaborolan-2-yl)phenoxy]butanoate as a colorless oil, (1.17 g, 35%). ¹H NMR (CDCl₃), δ7.73 (m, 2H), 6.87 (m, 2H), 4.14 (q, J=7.1 Hz, 2H), 4.03 (t, J=6.1 Hz, 2H), 2.51 (t, J=7.3 Hz, 2H), 2.11 (m, 2H), 1.33 (s, 12H), 1.25 (t, J=7.1 Hz, 3H). Ethyl 4-[4-(4,4,5,5-tetramethyl-1,3,2-dioxaborolan-2-yl)phenoxy]butanoate (1.17 g, 3.50 mmol) was dissolved in ethanol (10 mL) and lithium hydroxide (1M, 10 mL) was added. The resulting mixture was stirred at ambient temperature for 1 h, then acidified with hydrochloric acid (1M, 25.0 mL). The aqueous mixture was extracted with EtOAc (3×20 mL) and the combined organic extracts were dried over sodium sulfate and concentrated in vacuo to leave 4-[4-(4,4,5,5-tetramethyl-1,3,2-dioxaborolan-2-yl)phenoxy]butanoic acid as a white solid (0.906 g, 96%). ¹H NMR (CDCl₃), δ 7.65 (m, 2H), 6.89 (m, 2H), 4.04 (t, J=6.2 Hz, 2H), 2.48 (t, J=7.3 Hz, 2H), 2.06 (m, 2H), 1.32 (s, 12H).

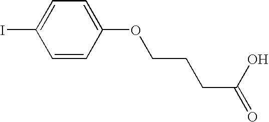

4-(4-iodophenoxy)butyric acid was also prepared from 4-iodophenol and ethyl 4-bromobutanoate by the same methodology used to prepare 4-[4-(4,4,5,5-tetramethyl-1,3,2-dioxaborolan-2-yl)phenoxy]butanoic acid. ¹H NMR (CD₃OD), δ 7.54 (m, 2H), 6.73 (m, 2H), 3.99 (t, J=6.3 Hz, 2H), 2.47 (t, J=7.3 Hz, 2H), 204 (m, 2H).

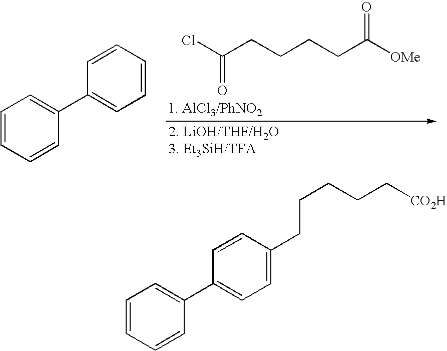

6-(4-phenylphenyl)hexanoic acid: Methyl 5-(chlorocarbonyl)pentanoate (2.00 g, 11.2 mmol) was added to a cooled (˜5° C.) solution of phenylbenzene (biphenyl) (0.580 g, 3.73 mmol) in nitrobenzene (15 mL). Aluminium trichloride (2.99 g, 22.4 mmol) was added portionwise to the reaction mixture, stirring was continued for 16 h at ambient temperature, and then the mixture was poured on to crushed ice (50 mL). The reaction mixture was allowed to stand for 1 h, the biphasic mixture separated, and the aqueous phase was extracted with dichloromethane. The combined organic extracts were washed with hydrochloric acid (2×, 0.1 M), then sodium carbonate solution (2×, 5%) and dried over magnesium sulfate and concentrated in vacuo to leave a pale tan solid. The crude product was dissolved in tetrahydrofuran (10 mL) and lithium hydroxide (0.82 M, 5 mL) was added and the mixture was stirred for 2 h. The reaction mixture was concentrated in vacuo, and the aqueous residue was extracted with dichloromethane then EtOAc, then acidified with hydrochloric acid (0.1 M). The aqueous phase was further extracted with EtOAc (3×), the combined organic extracts were dried over magnesium sulfate and concentrated in vacuo to give 6-oxo-6-(4-phenylphenyl)hexanoic acid as a cream colored solid (0.220 g, 21%). ESMS m/e: 283.2 (M−H)⁻; ¹H NMR (CDCl₃), δ 8.03 (m, 2H), 7.68 (m, 2H), 7.63 (m, 2H), 7.47 (m, 2H) 7.40 (m, 1H), 3.04 (t, J=7.0 Hz, 2H), 2.44 (t, J=7.2 Hz, 2H), 1.84 (m, 2H), 1.77 (m, 2H). 6-Oxo-6-(4-phenylphenyl)hexanoic acid (0.220 g, 0.780 mmol) was dissolved in trifluoroacetic acid (5 mL) and triethylsilane (0.310 mL, 1.25 mmol) was added dropwise. The reaction mixture was heated at 55° C., under nitrogen for 72 h, then cooled to ambient temperature and concentrated in vacuo to leave an oily solid. The crude product was partitioned between saturated sodium bicarbonate solution and diethyl ether. The aqueous layer was acidified with hydrochloric acid, extracted with diethyl ether (3×10 mL), the combined organic extracts were dried over magnesium sulfate and concentrated in vacuo to leave 6-(4-phenylphenyl)hexanoic acid as a pale tan solid (105 mg, 50%). ESMS m/e: 267.0 (M−H)⁻; ¹H NMR (CDCl₃), δ 7.58 (m, 2H), 7.51 (m, 2H), 7.42 (m, 2H), 7.32 (m, 1H) 7.24 (m, 2H), 2.66 (t, J=7.7 Hz, 2H), 2.37 (t, J=7.5 Hz, 2H), 1.68 (m, 4H), 1.43 (m, 2H).

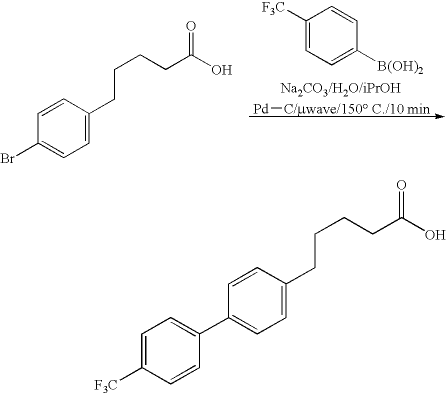

5-{4-[4-(Trifluoromethyl)phenyl]phenyl}pentanoic acid: A mixture of 5-(4-bromophenyl)pentanoic acid (300 mg, 1.17 mmol), 4-(trifluoromethyl)-benzeneboronic acid (243 mg, 1.28 mmol), sodium carbonate (154 mg, 1.41 mmol), water (1.55 mL) and isopropanol (0.220 mL) was heated at 150° C. for 10 min. The reaction mixture was partitioned between water and EtOAc, the organic phase was separated, dried over magnesium sulfate and concentrated in vacuo to give 5-{4-[4-(trifluoromethyl)phenyl]phenyl}pentanoic acid as a solid (120 mg, 32%). ESMS m/e: 321.3 (M−H)⁻; ¹H NMR (CDCl₃), δ 7.67 (s, 4H), 7.51 (d, J=8.2 Hz, 2H), 7.27 (d, J=8.2 Hz, 2H), 2.70 (m, 2H), 2.41 (m, 2H), 1.72 (m, 4H).

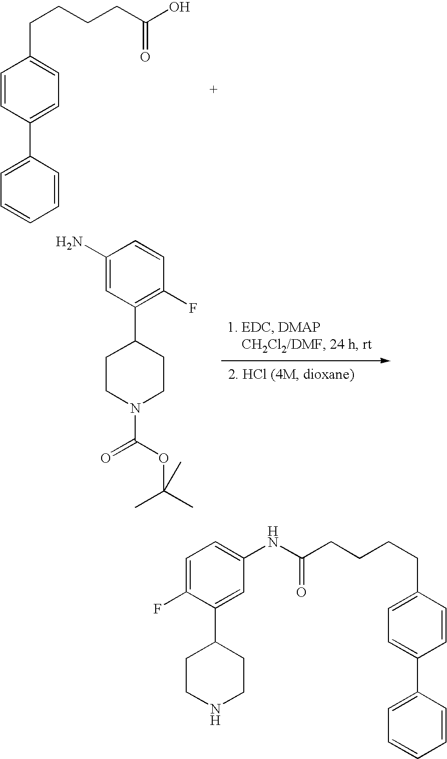

N-(4-fluoro-3-(4-piperidyl)phenyl)-5-(4-phenylphenyl)pentanamide: 5-(4-biphenylyl)valeric acid (0.20 mmol, 50 mg), EDC (100 mg, 0.52 mmol), DMAP (15 mg, 0.12 mmol) in 2 mL of CH₂Cl₂/DMF (10:1) were stirred for 15 minutes followed by addition of tert-butyl 4-(5-amino-2-fluorophenyl)-1-piperidinecarboxylate (0.17 mmol, 50 mg). The reaction mixture was stirred at room temperature for 24 h. The reaction mixture was applied directly to a preparative TLC (without any workup) (silica gel, 1:1 hexane/EtOAc) to afford the tert-butyl 4-{2-fluoro-5-[5-(4-phenylphenyl)pentanoylamino]phenyl}piperidinecarboxylate. ¹H NMR (400 MHz, CDCl₃) δ 7.60-7.55 (m, 2H), 7.54-7.48 (m, 2H), 7.45-7.39 (m, 2H), 7.39-7.28 (m, 4H), 7.27-7.22 (m, 2H), 6.98-6.91 (m, 1H), 4.33-4.13 (br, 2H), 3.01-2.90 (m, 1H), 2.89-2.60 (m, 2H), 2.69 (t, 2H, J=7.2 Hz), 2.37 (t, 2H, J=7.2 Hz), 1.85-1.04 (m, 9H); ESI-MS m/e: 529.5 (M−H)⁻. The tert-butyl 4-{2-fluoro-5-[5-(4-phenylphenyl)pentanoyl amino]phenyl}piperidinecarboxylate from the previous step was treated with 4 M HCl in dioxane(1 mL) at room temperature for 2 h. Removal of solvent in vacuo and purification on a silica TLC (silica gel, 2M NH₃/MeOH:EtOAc, 1:4) (11.6 mg, 15% in two steps) afforded N-(4-fluoro-3-(4-piperidyl)phenyl)-5-(4-phenylphenyl) pentanamide. ¹H NMR (400 MHz, CDCl₃) δ 8.51 (s, 1H), 7.76-7.69 (m, 1H), 7.58-7.52 (m, 2H), 7.51-7.45 (m, 2H), 7.45-7.36 (m, 2H), 7.35-7.27 (m, 1H), 7.25-7.18 (m, 3H), 6.97-6.89 (m, 1H), 3.55-3.44 (m, 2H), 3.08-2.97 (m, 1H), 2.97-2.86 (m, 2H). 2.66 (t, 2H, J=7.2 Hz), 2.46 (t, 2H, J=7.2Hz), 2.00-1.83 (m, 4H), 1.83-1.65 (m, 4H); ESI-MS m/e: 431.3 (M+H)⁺.

The following compounds were prepared analogously:

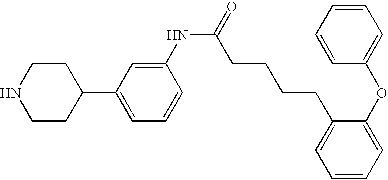

5-(2-phenoxyphenyl)-N-(3-(4-piperidyl)phenyl)pentanam ide: Prepared according to the procedures outlined in Scheme 9. ESI-MS m/e: 429.4 (M+H)⁺.

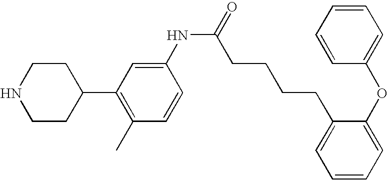

N-(4-Methyl-3-(4-piperidyl)phenyl)-5-(2-phenoxyphenyl)pentanamide: Prepared according to the procedures outlined in Scheme 9. ESI-MS m/e: 443.4 (M+H)⁺.

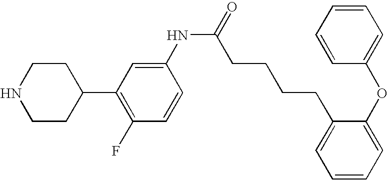

N-(4-Fluoro-3-(4-piperidyl)phenyl)-5-(2-phenoxyphenyl)pentanamide: Prepared according to the procedures outlined in Scheme 9. ESI-MS m/e: 447.3 (M+H)⁺.

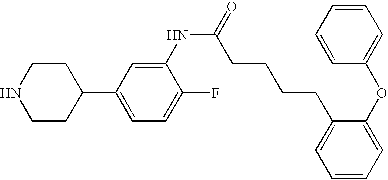

N-(2-Fluoro-5-(4-piperidyl)phenyl)-5-(2-phenoxyphenyl)pentanamide: Prepared according to the procedures outlined in Scheme 9. ESI-MS m/e: 447.3 (M+H)⁺.

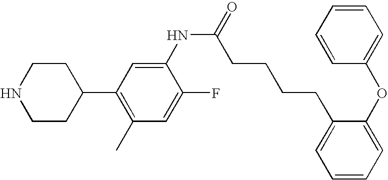

N-(2-Fluoro-4-methyl-5-(4-piperidyl)phenyl)-5-(2-phenoxyphenyl)pentanamide: Prepared according to the procedures outlined in Scheme 9. ESI-MS m/e: 461.3 (M+H)⁺.

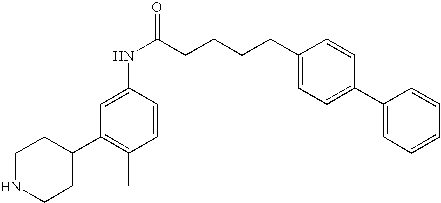

N-(4-Methyl-3-(4-piperidyl)phenyl)-5-(4-phenylphenyl)pentanamide: Prepared according to the procedures outlined in Scheme 9. ESI-MS m/e: 427.3 (M+H)⁺.

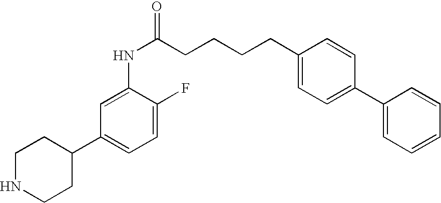

N-(2-Fluoro-5-(4-piperidyl)phenyl)-5-(4-phenylphenyl)pentanamide: Prepared according to the procedures outlined in Scheme 9. ESI-MS m/e: 431.3 (M+H)⁺.

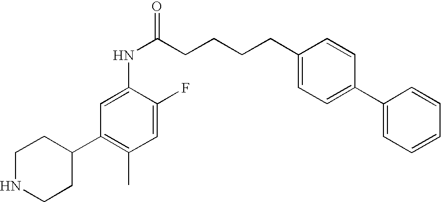

N-(2-Fluoro-4-methyl-5-(4-piperidyl)phenyl)-5-(4-phenylphenyl)pentanamide: Prepared according to the procedures outlined in Scheme 9. ESI-MS m/e: 445.3 (M+H)⁺.

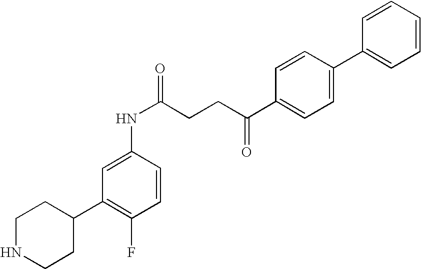

N-(4-fluoro-3-(4-piperidyl)phenyl)-4-oxo-4-(4-phenylphenyl)butanamide: Prepared according to the procedures outlined in Scheme 9. ESI-MS m/e: 431.3 (M+H)⁺.

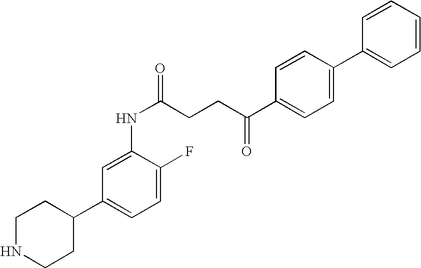

N-(2-fluoro-5-(4-piperidyl)phenyl)-4-oxo-4-(4-phenylphenyl)butanamide: Prepared according to the procedures outlined in Scheme 9. ESI-MS m/e: 431.3 (M+H)⁺.

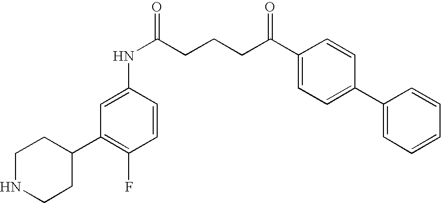

N-(4-fluoro-3-(4-piperidyl)phenyl)-5-oxo-5-(4-phenylphenyl)pentanamide: Prepared according to the procedures outlined in Scheme 9. ESI-MS m/e: 445.3 (M+H)⁺.

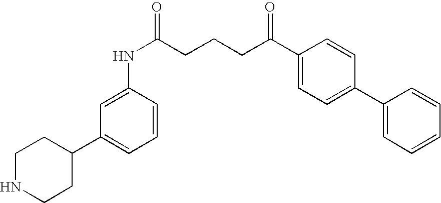

5-oxo-5-(4-phenylphenyl)-N-(3-(4-piperidyl)phenyl)pentanamide: Prepared according to the procedures outlined in Scheme 9. ESI-MS m/e: 427.3 (M+H)⁺.

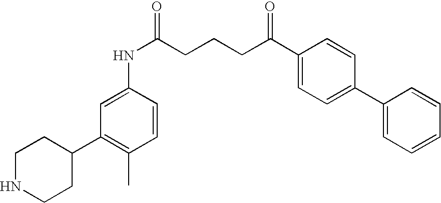

N-(4-methyl-3-(4-piperidyl)phenyl)-5-oxo-5-(4-phenylphenyl)pentanamide: Prepared according to the procedures outlined in Scheme 9. ESI-MS m/e: 441.3 (M+H)⁺.

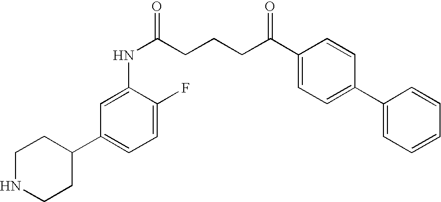

N-(2-fluoro-5-(4-piperidyl)phenyl)-5-oxo-5-(4-phenylphenyl)pentanamide: Prepared according to the procedures outlined in Scheme 9. ESI-MS m/e: 445.2 (M+H)⁺.

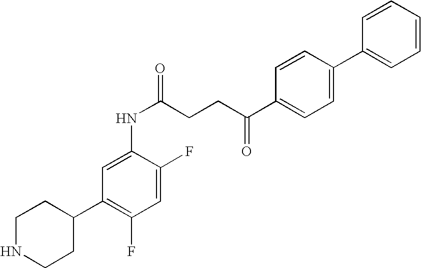

N-(2,4-difluoro-5-(4-piperidyl)phenyl)-4-oxo-4-(4-phenylphenyl)butanamide: Prepared according to the procedures outlined in Scheme 9. ESI-MS m/e: 449.2 (M+H)⁺.

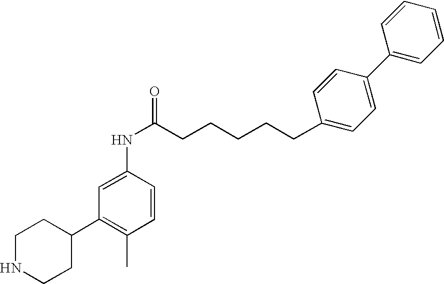

N-(4-methyl-3-(4-piperidyl)phenyl)-6-(4-phenylphenyl)hexanamide Prepared according to the procedures outlined in Scheme 9. ESI-MS m/e: 441.3 (M+H)⁺.

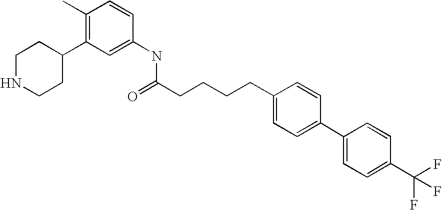

N-(4-methyl-3-(4-piperidyl)phenyl)-5-{4-[4-(trifluoromethyl)phenyl]phenyl}pentanamide: Prepared according to the procedures outlined in Scheme 9. ESI-MS m/e: 495.1 (M+H)⁺.

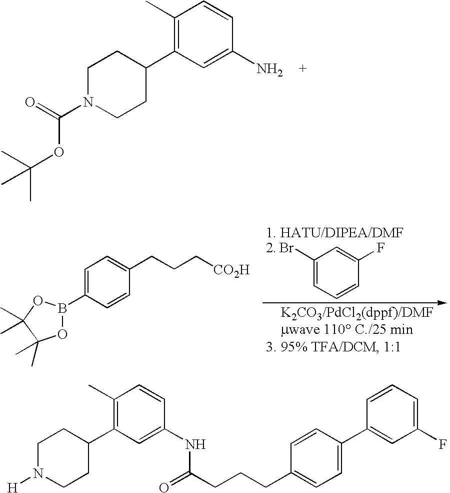

4-[4-(3-fluorophenyl)phenyl]-N-(4-methyl-3-(4-piperidyl)phenyl)butanamide: A mixture of tert-butyl 4-(3-amino-6-methylphenyl)piperidinecarboxylate (39.6 mg, 0.130 mmol), 4-[4-(4,4,5,5-tetramethyl-1,3,2-dioxaborolan-2-yl)phenyl]butanoic acid, (38.0 mg, 0.130 mmol), [dimethylamino-([1,2,3]triazolo[4,5-b]pyridin-3-yloxy)-methylene]-dimethyl-ammonium hexafluoro phosphate (HATU, 54.3 mg, 0.140 mmol) and diisopropylethylamine (68.0 μL, 0.390 mmol) in dimethylformamide (2 mL) was stirred at ambient temperature for 18 h. The reaction mixture was concentrated in vacuo, dissolved in EtOAc (20 mL) and washed with sodium bicarbonate (saturated, 10 mL) and water (2×10 mL) then dried over sodium sulfate and concentrated in vacuo to give a gum. Purification on silica gel (silica gel 60, 40 mL) eluting with cyclohexane:EtOAc, 8:1, then 4:1 gave tert-butyl 4-(2-methyl-5-{4-[4-(4,4,5,5-tetramethyl(1,3,2-dioxaborolan-2-yl))phenyl]butanoylamino}phenyl)piperidinecarboxylate as a yellow oil (39.0 mg, 53%). ESI-MS m/e: 563.3 (M+H)⁺. A degassed solution of tert-butyl 4-(2-methyl-5-{4-[4-(4,4,5,5-tetramethyl(1,3,2-dioxaborolan-2-yl))phenyl]butanoylamino}phenyl)piperidine carboxylate (19.0 mg, 0.033 mmol) in dimethylformamide (0.8 mL), was added to a mixture of 3-fluorobromobenzene (6.55 mg, 0.370 mmol), 1,1′-Bis-(diphenylphosphino)-ferrocenedichloropalladium (3.30 mg, 8 mol %) and cesium carbonate solution,(2M, 50 μL) and the resulting mixture was heated in the microwave at 110° C. for 25 min. The reaction mixture was concentrated in vacuo, then partitioned between water (20 mL) and EtOAc (2×10 mL). The organic phase was separated, washed with water (2×20 mL), dried over sodium sulfate and concentrated in vacuo to leave a gum. Purification of the crude product on silica gel (silica gel 60, 20 mL) eluting with cyclohexane:EtOAc, 85:15 then 4:1 gave tert-butyl 4-(5-{4-[4-(3-fluorophenyl)phenyl]butanoylamino}-2-methylphenyl)piperidine carboxylate (13.0 mg, 73%). ESI-MS m/e: 431.3 (M-C₅H₈O₂+H)⁺. The resulting tert-butyl 4-(5-{4-[4-(3-fluorophenyl)phenyl]butanoylamino}-2-methylphenyl)piperidine carboxylate was dissolved in dichloromethane (0.8 mL) and trifluoracetic acid (95%, 0.8 mL) was added and reaction mixture was stirred for 2 h, then concentrated in vacuo. The residue was redissolved in acetonitrile (1 mL), hydrochloric acid (1M, 1 mL) was added, and the reaction mixture was concentrated in vacuo to give 4-[4-(3-fluorophenyl)phenyl]-N-(4-methyl-3-(4-piperidyl)phenyl)butanamide hydrochloride as a white solid (11.2 mg, 98%). ESMS m/e: 431.2 (M+H)+; ¹H NMR (CD₃OD) δ 7.59 (m, 1H), 7.53 (d, J=8.0 Hz, 2H), 7.40 (m, 2H) 7.30 (m, 3H), 7.17 (m, 1H), 7.10 (d, J=8.4 Hz, 1H), 7.03 (m, 1H), 3.49 (d, J=12.6 Hz, 2H), 3.17 (td, J=12.7, 2.8 Hz, 2H), 3.11 (tt, J=12.0, 3.5 Hz, 1H), 2.75 (t, J=7.5 Hz, 2H), 2.41 (t, J=7.5 Hz, 2H), 2.32 (s, 3H), 2.05 (quartet, J=7.5 Hz, 2H), 1.93 (m, 4H).

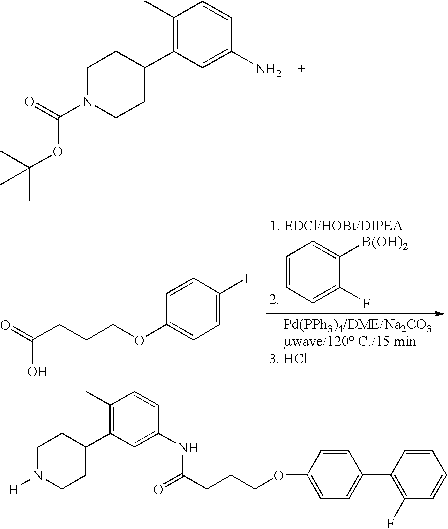

4-[4-(2-fluorophenyl)phenoxy]-N-(4-methyl-3-(4-piperidyl)phenyl)butanamide: A mixture of tert-butyl 4-(3-amino-6-methylphenyl)piperidinecarboxylate (436 mg, 1.50 mmol), 4-(4-iodophenoxy)butanoic acid (460 mg, 1.50 mmol) 1-[3-dimethylaminopropyl]-3-ethylcarbodiimide hydrochoride (370 mg, 1.90 mmol), and HOBt (320 mg, 2.30 mmol) in dimethylformamide (10 mL) was stirred at ambient temperature for 2 h. The reaction mixture was partitioned between water (80 mL) and diethylether (3×20 mL). The organic phase was dried over sodium sulfate and concentrated in vacuo to leave a yellow oil. Purification of the crude product on an SPE cartridge (5 g) eluting with pentane followed by pentane:diethylether, 1:1, then 1:4 then 100% diethylether gave tert-butyl 4-{5-[4-(4-iodophenoxy)butanoylamino]-2-methylphenyl}piperidinecarboxylate as a cream colored foam (514 mg, 59%). ESMS m/e: 579.2 (M+H)⁺; ¹H NMR (CDCl₃) δ 7.54 (m, 2H), 7.30 (d, J=2.1 Hz, 1H), 7.23 (dd, J=8.2, 2.1 Hz, 1H), 7.17 (br s, 1H), 7.09 (d, J=8.2 Hz, 1H), 6.67 (m, 2H), (br s, 2H), 4.03 (t, J=5.9 Hz, 2H), 2.80 (m, 3H), 2.55 (t, J=7.1 Hz, 2H), 2.30 (s, 3H), 2.20 (m, 2H), 1.73 (br d, J=13.6 Hz, 2H), 1.58 (m, 2H), 1.49 (s, 9H). tert-Butyl 4-{5-[4-(4-iodophenoxy)butanoylamino]-2-methylphenyl}piperidinecarboxylate and 2-fluorobenzeneboronic acid (35 mg, 0.250 mmol), tetrakis triphenylphosphine palladium (0) (12.0 mg, 10 mol %), aqueous sodium carbonate (2 M, 1 mL) and dimethoxyethane (2 mL) was heated in the microwave at 120° C. for 15 min. The reaction mixture was diluted with water (2 mL), extracted with ethyl acetate (3×0.5 mL) and the combined organic phases were blown down using nitrogen. The resulting gum was purified on an SPE cartridge (5 g) eluting with cyclohexane:ethyl acetate, 9:1 then 3:2 to give the tert-butyl 4-(5-{4-[4-(2-fluorophenyl)phenoxy]butanoylamino}-2-methylphenyl)piperidinecarboxylate (60.0 mg, quantitative). ESMS m/e: 447.3 (M-C₅H₈O₂+H)⁺; ¹H NMR (CDCl₃) δ 7.47 (2H, m), 7.40 (1H, td, J=7.7, 1.9 Hz), 7.34 (1H, d, J=1.8 Hz), 7.28 (1H, m), 7.24 (2H, m) 7.18 (1H, td, J=7.7, 1.2 Hz), 7.13 (1H, ddd, J=10.8, 8.1, 1.2 Hz), 7.09 (1H, d, J=8.2 Hz), 6.97 (2H, m), 4.26 (2H, broad s ), 4.11 (2H, t, J=6.0 Hz), 2.80 (3H, m), 2.59 (2H, t, J=7.1 Hz), 2.30 (3H, s), 2.24 (2H, m), 1.73 (2H, broad d), J=12.3 Hz), 1.60 (2H, qd, J=12.3, 3.8 Hz), 1.48 (9H, s). The amide was dissolved in dichloromethane (1 mL) and trifluoroacetic acid (1 mL) was added. The resulting solution was orbitally shaken for 1 h, then blown down using nitrogen to leave an oil. Purification on an amino SPE cartridge (2 g) eluting with diethylether, followed by ethyl acetate, then 10% methanol in ethyl acetate gave 4-[4-(2-fluorophenyl)phenoxy]-N-(4-methyl-3-(4-piperidyl)phenyl)butanamide. This was dissolved in methanol (2 mL) and hydrochloric acid (1M, 3 mL) was added and the resulting mixture was concentrated in vacuo and triturated with diethylether to give 4-[4-(2-fluorophenyl)phenoxy]-N-(4-methyl-3-(4-piperidyl)phenyl)butanamide. ESMS m/e: 447.2 (M+H)⁺; ¹H NMR (DMSO-D₆) δ 9.95 (s, 1H), 8.92 (br d, J=10.9 Hz, 1H), 8.68 (br q, J=10.9 Hz, 1H,), 7.58 (d, J=2.0 Hz, 1H) 7.49 (m, 3H), 7.37 (m, 1H), 7.28 (m, 3H), 7.07 (d, J=8.6 Hz, 1H), 7.04 (m, 2H), 4.07 (t, J=6.4 Hz, 2H), 3.36 (4H, under water peak), 3.02 (m, 3H), 2.25 (s, 3H), 2.05 (quintet, 2H), 1.81 (m, 4H).

The following compounds were prepared analogously:

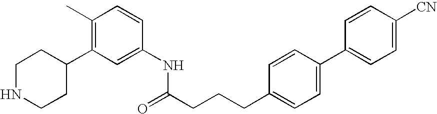

4-[4-(4-cyanophenyl)phenyl]-N-(4-methyl-3-(4-piperidyl)phenyl)butanamide: Prepared according to the procedures outlined in Scheme 11. ESI-MS m/e: 438.0 (M+H)⁺.

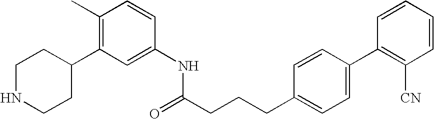

4-[4-(2-cyanophenyl)phenyl]-N-(4-methyl-3-(4-piperidyl)phenyl)butanamide: Prepared according to the procedure outlined in Scheme 11. ESI-MS m/e: 438.0 (M+H)⁺.

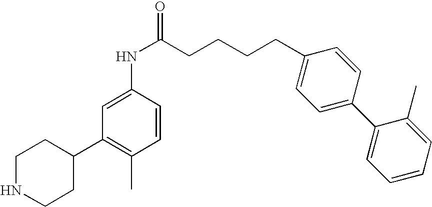

N-(4-methyl-3-(4-piperidyl)phenyl)-5-[4-(2-methylphenyl)phenyl]pentanamide: Prepared according to the procedures outlined in Scheme 11. ESMS m/e: 441.3 (M+H)⁺.

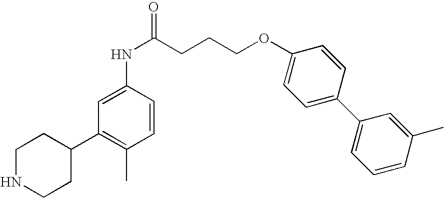

N-(4-methyl-3-(4-piperidyl)phenyl)-4-[4-(3-methylphenyl)phenoxy]butanamide: Prepared according to the procedures outlined in Scheme 11. ESMS m/e: 443.1 (M+H)⁺.

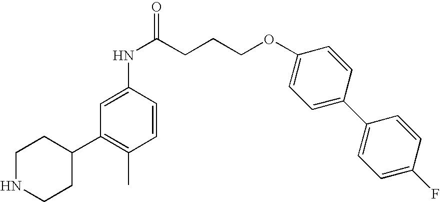

4-[4-(4-fluorophenyl)phenoxy]-N-(4-methyl-3-(4-piperidyl)phenyl)butanamide: Prepared according to the procedure outlined in Scheme 11. ESMS m/e: 447.2 (M+H)⁺.

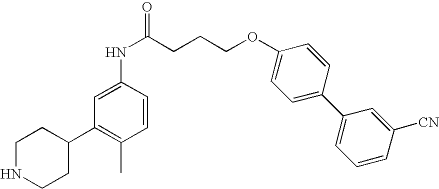

4-[4-(3-cyanophenyl)phenoxy]-N-(4-methyl-3-(4-piperidyl)phenyl)butanamide: Prepared according to the procedure outlined in Scheme 11. ESMS m/e: 454.2 (M+H)⁺.

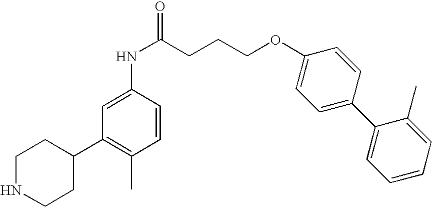

N-(4-methyl-3-(4-piperidyl)phenyl)-4-[4-(2-methylphenyl)phenoxy]butanamide: Prepared according to the procedures outlined in Scheme 11. ESMS mle: 443.2 (M+H)⁺.

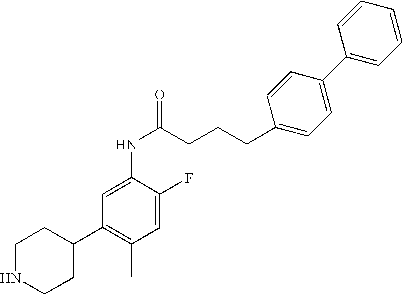

N-(2-fluoro-4-methyl -5-(4-piperidyl)phenyl)-4-(4-phenylphenyl)butanamide: Prepared according to the procedures outlined in Scheme 11. ESMS m/e: 431.3 (M+H)⁺.

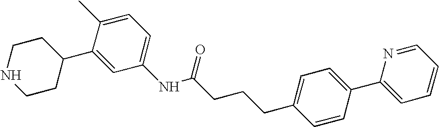

N-(4-methyl-3-(4-piperidyl)phenyl)-4-(4-(2-pyridyl)phenyl)butanamide: Prepared according to the procedures outlined in Scheme 11. ESMS m/e: 414.2 (M+H)⁺.

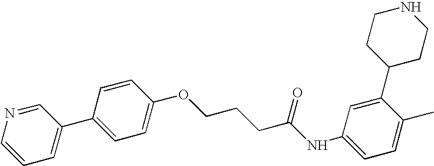

N-(4-methyl-3-(4-piperidyl)phenyl-4-(4-(3-pyridyl)phenoxy)butanamide: Prepared according to the procedures outlined in Scheme 11. ESMS m/e: 430.3 (M+H)⁺.

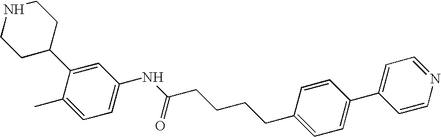

N-(4-methyl-3-(4-piperidyl)phenyl)-5-(4-(4-pyridyl)phenyl)pentanamide: Prepared according to the procedures outlined in Scheme 11. ESMS m/e: 428.3 (M+H)⁺.

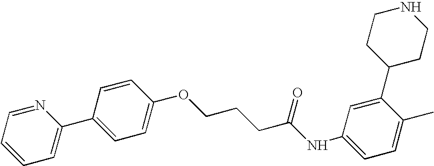

N-(4-methyl-3-(4-piperidyl)phenyl)-4-(4-(2-pyridyl)phenoxy)butanamide: Prepared according to the procedures outlined in Scheme 11. ESMS m/e: 430.1 (M+H)⁺.

### III. ORAL COMPOSITIONS

As a specific embodiment of an oral composition of a compound of this invention, 100 mg of one of the compounds described herein is formulated with sufficient finely divided lactose to provide a total amount of 580 to 590 mg to fill a size 0 hard gel capsule.

### IV. PHARMACOLOGICAL EVALUATION OF COMPOUNDS AT CLONED RAT MCH1 RECEPTOR

The pharmacological properties of the compounds of the present invention were evaluated at the cloned rat MCH1 receptor using the protocols described below.

Host Cells

A broad variety of host cells can be used to study heterologously expressed proteins. These cells include, but are not restricted to, assorted mammalian lines such as: Cos-7, CHO, LM(tk−), HEK293 and Peak rapid 293; insect cell lines such as Sf9 and Sf21; amphibian cells such as xenopus oocytes; and others. COS 7 cells are grown on 150 mm plates in DMEM with supplements (Dulbecco's Modified Eagle Medium with 10% bovine calf serum, 4 mM glutamine, 100 units/ml penicillin/100 Fg/ml streptomycin) at 37° C., 5% CO₂. Stock plates of COS-7 cells are trypsinized and split 1:6 every 3-4 days. Human embryonic kidney 293 cells are grown on 150 mm plates in DMEM with supplements (10% bovine calf serum, 4 mM glutamine, 100 units/ml penicillin/100 Fg/ml streptomycin) at 37° C., 5% CO₂. Stock plates of 293 cells are trypsinized and split 1:6 every 3-4 days.

Human embryonic kidney Peak rapid 293 (Peakr293) cells are grown on 150 mm plates in DMEM with supplements (10% fetal bovine serum, 10% L-glutamine, 50 Fg/ml gentamycin) at 37° C., 5% CO₂. Stock plates of Peak rapid 293 cells are trypsinized and split 1:12 every 3-4 days. Mouse fibroblast LM(tk−) cells are grown on 150 mm plates in DMEM with supplements (Dulbecco's Modified Eagle Medium with 10% bovine calf serum, 4 mM glutamine, 100 units/ml penicillin/100 Fg/ml streptomycin) at 37° C., 5% CO₂. Stock plates of LM(tk−) cells are trypsinized and split 1:10 every 3-4 days. Chinese hamster ovary (CHO) cells were grown on 150 mm plates in HAM's F-12 medium with supplements (10% bovine calf serum, 4 mM L-glutamine and 100 units/ml penicillin/100 Fg/ml streptomycin) at 37° C., 5% CO₂. Stock plates of CHO cells are trypsinized and split 1:8 every 3-4 days. Mouse embryonic fibroblast NIH-3T3 cells are grown on 150 mm plates in Dulbecco's Modified Eagle Medium (DMEM) with supplements (10% bovine calf serum, 4 mM glutamine, 100 units/ml penicillin/100 Fg/ml streptomycin) at 37° C., 5% CO₂. Stock plates of NIH-3T3 cells are trypsinized and split 1:15 every 3-4 days. Sf9 and Sf21 cells are grown in monolayers on 150 mm tissue culture dishes in TMN-FH media supplemented with 10% fetal calf serum, at 27° C., no CO₂. High Five insect cells are grown on 150 mm tissue culture dishes in Ex-Cell ₄₀₀TM medium supplemented with L-Glutamine, also at 27° C., no CO₂. In some cases, cell lines that grow as adherent monolayers can be converted to suspension culture to increase cell yield and provide large batches of uniform assay material for routine receptor screening projects.

Transient Expression

DNA encoding proteins to be studied can be transiently expressed in a variety of mammalian, insect, amphibian and other cell lines by several methods including, but not restricted to, calcium phosphate-mediated, DEAE-dextran mediated, Liposomal-mediated, viral-mediated, electroporation-mediated and microinjection delivery. Each of these methods may require optimization of assorted experimental parameters depending on the DNA, cell line, and the type of assay to be subsequently employed. A typical protocol for the calcium phosphate method as applied to Peak rapid 293 cells is described as follows: Adherent cells are harvested approximately twenty-four hours before transfection and replated at a density of 3.5×10⁶ cells/dish in a 150 mm tissue culture dish and allowed to incubate over night at 37° C. at 5% CO₂. 250 Fl of a mixture of CaCl₂ and DNA (15 Fg DNA in 250 mM CaCl₂) is added to a 5 ml plastic tube and 500 Fl of 2×HBS (280 mM NaCl, 10 mM KCl, 1.5 mM Na₂HPO₄, 12 mM dextrose, 50 mM HEPES) is slowly added with gentle mixing. The mixture is allowed to incubate for 20 minutes at room temperature to allow a DNA precipitate to form. The DNA precipitate mixture is then added to the culture medium in each plate and incubated for 5 hours at 37° C., 5% CO₂. After the incubation, 5 ml of culture medium (DMEM, 10% FBS, 10% L-glut and 50 μg/ml gentamycin) is added to each plate. The cells are then incubated for 24 to 48 hours at 37° C., 5% CO₂. A typical protocol for the DEAE-dextran method as applied to Cos-7 cells is described as follows; Cells to be used for transfection are split 24 hours prior to the transfection to provide flasks which are 70-80% confluent at the time of transfection. Briefly, 8 Fg of receptor DNA plus 8 Fg of any additional DNA needed (e.g. G_{α} protein expression vector, reporter construct, antibiotic resistance marker, mock vector, etc.) are added to 9 ml of complete DMEM plus DEAE-dextran mixture (10 mg/ml in PBS). Cos-7 cells plated into a T225 flask (sub-confluent) are washed once with PBS and the DNA mixture is added to each flask. The cells are allowed to incubate for 30 minutes at 37° C., 5% CO₂. Following the incubation, 36 ml of complete DMEM with 80 FM chloroquine is added to each flask and allowed to incubate an additional 3 hours. The medium is then aspirated and 24 ml of complete medium containing 10% DMSO for exactly 2 minutes and then aspirated. The cells are then washed 2 times with PBS and 30 ml of complete DMEM added to each flask. The cells are then allowed to incubate over night. The next day the cells are harvested by trypsinization and reseeded as needed depending upon the type of assay to be performed.

A typical protocol for liposomal-mediated transfection as applied to CHO cells is described as follows; Cells to be used for transfection are split 24 hours prior to the transfection to provide flasks which are 70-80% confluent at the time of transfection. A total of 10 Fg of DNA which may include varying ratios of receptor DNA plus any additional DNA needed (e.g. G_{α} protein expression vector, reporter construct, antibiotic resistance marker, mock vector, etc.) is used to transfect each 75 cm² flask of cells. Liposomal mediated transfection is carried out according to the manufacturer's recommendations (LipofectAMINE, GibcoBRL, Bethesda, Md.). Transfected cells are harvested 24 hours post transfection and used or reseeded according to the requirements of the assay to be employed. A typical protocol for the electroporation method as applied to Cos-7 cells is described as follows; Cells to be used for transfection are split 24 hours prior to the transfection to provide flasks which are subconfluent at the time of transfection. The cells are harvested by trypsinization resuspended in their growth media and counted. 4×10⁶ cells are suspended in 300 Fl of DMEM and placed into an electroporation cuvette. 8 Fg of receptor DNA plus 8 Fg of any additional DNA needed (e.g. G_{α} protein expression vector, reporter construct, antibiotic resistance marker, mock vector, etc.) is added to the cell suspension, the cuvette is placed into a BioRad Gene Pulser and subjected to an electrical pulse (Gene Pulser settings: 0.25 kV voltage, 950 FF capacitance). Following the pulse, 800 Fl of complete DMEM is added to each cuvette and the suspension transferred to a sterile tube. Complete medium is added to each tube to bring the final cell concentration to 1×10⁵ cells/100 Fl. The cells are then plated as needed depending upon the type of assay to be performed.

A typical protocol for viral mediated expression of heterologous proteins is described as follows for baculovirus infection of insect Sf9 cells. The coding region of DNA encoding the receptor disclosed herein may be subcloned into pBlueBacIII into existing restriction sites or sites engineered into sequences 5′ and 3′ to the coding region of the polypeptides. To generate baculovirus, 0.5 Fg of viral DNA (BaculoGold) and 3 Fg of DNA construct encoding a polypeptide may be co-transfected into 2×106 Spodoptera frugiperda insect Sf9 cells by the calcium phosphate co-precipitation method, as outlined in by Pharmingen (in “Baculovirus Expression Vector System: Procedures and Methods Manual”). The cells then are incubated for 5 days at 27° C. The supernatant of the co-transfection plate may be collected by centrifugation and the recombinant virus plaque purified. The procedure to infect cells with virus, to prepare stocks of virus and to titer the virus stocks are as described in Pharmingen's manual. Similar principals would in general apply to mammalian cell expression via retro-viruses, Simliki forest virus and double stranded DNA viruses such as adeno-, herpes-, and vacinia-viruses, and the like.

Stable Expression

Heterologous DNA can be stably incorporated into host cells, causing the cell to perpetually express a foreign protein. Methods for the delivery of the DNA into the cell are similar to those described above for transient expression but require the co-transfection of an ancillary gene to confer drug resistance on the targeted host cell. The ensuing drug resistance can be exploited to select and maintain cells that have taken up the heterologous DNA. An assortment of resistance genes are available including, but not restricted to, Neomycin, Kanamycin, and Hygromycin. For the purposes of receptor studies, stable expression of a heterologous receptor protein is carried out in, but not necessarily restricted to, mammalian cells including, CHO, HEK293, LM(tk−), etc.

Cell Membrane Preparation

For binding assays, pellets of transfected cells are suspended in ice-cold buffer (20 mM Tris.HCl, 5 mM EDTA, pH 7.4) and homogenized by sonication for 7 sec. The cell lysates are centrifuged at 200×g for 5 min at 4° C. The supernatants are then centrifuged at 40,000×g for 20 min at 4° C. The resulting pellets are washed once in the homogenization buffer and suspended in binding buffer (see methods for radioligand binding). Protein concentrations are determined by the method of Bradford (1976) using bovine serum albumin as the standard. Binding assays are usually performed immediately, however it is possible to prepare membranes in batch and store frozen in liquid nitrogen for future use.

Radioligand Binding Assays

Radioligand binding assays for the rat MCH1 receptor were carried out using plasmid pcDNA3.1-rMCH1-f (ATCC Patent Deposit Designation No. PTA-3505). Plasmid pcDNA3.1-rMCH1-f comprises the regulatory elements necessary for expression of DNA in a mammalian cell operatively linked to DNA encoding the rat MCH1 receptor so as to permit expression thereof. Plasmid pcDNA3.1-rMCH1-f was deposited on Jul. 5, 2001, with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure and was accorded ATCC Patent Deposit Designation No. PTA-3505. Binding assays can also be performed as described hereinafter using plasmid pEXJ.HR-TL231 (ATCC Accession No. 203197) Plasmid pEXJ.HR-TL231 encodes the human MCH1 receptor and was deposited on Sep. 17, 1998, with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure and was accorded ATCC Accession No. 203197. Human embryonic kidney Peak rapid 293 cells (Peakr293 cells) were transiently transfected with DNA encoding the MCH1 receptor utilizing the calcium phosphate method and cell membranes were prepared as described above. Binding experiments with membranes from Peakr293 cells transfected with the rat MCH1 receptor were performed with 0.08 nM [³H]Compound A using an incubation buffer consisting of 50 mM Tris pH 7.4, 10 mM MgCl₂, 0.16 mM PMSF, 1 mM 1,10 phenantroline and 0.2% BSA. Binding was performed at 25° C. for 90 minutes. Incubations were terminated by rapid vacuum filtration over GF/C glass fiber filters, presoaked in 5% PEI using 50 nM Tris pH 7.4 as wash buffer. In all experiments, nonspecific binding is defined using 10 μM of TRITIATED METHYL(4S)-3-{[(3-{4-[3-(ACETYLAMINO)PHENYL]-1-PIPERIDINYL}PROPYL)AMINO]CARBONYL}-4-(3,4-DIFLUOROPHENYL)-6-(METHOXYMETHYL)-2-OXO-1,2,3,4-TETRAHYDRO-5-PYRIMIDINE CARBOXYLATE. The synthesis of this radiolabeled compound is described in WO 03/04027.

Binding Data

The above-identified assay was used to identify compounds of the instant invention as potent inhibitors of that MCH1 receptor. The Ki values for the disclosed compound range from 0.1 nM to 1000 nM. In one embodiment, the binding affinity for the compounds against the MCH1 receptor is from 0.5 nM to 500 nM. In one embodiment, the binding affinity for the compounds against the MCH1 receptor is from 1.0 nM to 100 nM. In another embodiment, the binding affinity for the compounds against the MCH1 receptor is from 1.0 nM to 75 nM.

### VI. IN-VIVO METHODS

A. Obesity

The following two (2) methods describe protocols which may be utilized to predict the efficacy of MCH1 antagonists for the treatment of obesity.

1. Effects of MCH1 Antagonists on Body Weight (3 Day)

Male Long Evans rats (Charles River) weighing 180-200 grams are housed in groups of four on a 12-hour light/dark cycle with free access to food and water. Test compounds are administered twice daily via i.p. injection, 1 hour before the dark cycle and 2 hours after lights on, for three days. All rats are weighed daily after each morning injection. Overall results are expressed as body weight (grams) gained per day (mean±SEM) and are analyzed by two-way ANOVA. Data for each time point are analyzed by one-way ANOVA followed by post hoc Newman-Keuls test. The data are then analyzed using the GraphPad Prism (v2.01) (GraphPad Software, Inc., San Diego, Calif.).

2. Effects of MCH1 Antagonists on Consumption of Sweetened Condensed Milk

Male C57BL/6 mice (Charles River) weighing 17-19 grams at the start of experiments are housed in groups of four or five on a 12 hour light/dark cycle with free access to food and water. For 7 days, mice are weighed, placed in individual cages and allowed to drink sweetened condensed milk (Nestle, diluted 1:3 with water) for 1 hour, 2-4 hours into the light cycle. The amount of milk consumed is determined by weighing the milk bottle before and after each drinking bout. On the test day, mice received i.p. injections of Test Compound (3, 10 or 30 mg/kg in 0.01% lactic acid), vehicle (0.01 % lactic acid) of d-fenfluramine (10 mg/kg in 0.01% lactic acid) 30 min. prior to exposure to milk. The amount of milk consumed on the test day (in mls milk/kg body weight) is compared to the baseline consumption for each mouse determined on the previous 2 days. Data for each time point are analyzed by one-way ANOVA.

B. Depression

The following method describes a protocol which may be used to predict the efficacy of MCH1 antagonists for the treatment of depression.

1. Forced Swim Test (FST) in the Rat

Animals

Male Sprague-Dawley rats (Taconic Farms, N.Y.) are used in all experiments. Rats are housed 5 per cage and maintained on a 12:12-h light-dark cycle. Rats are handled for 1 minute each day for 4 days prior to behavioral testing.

Drug Administration

Animals are randomly assigned to receive a single i.p. administration of vehicle (2.5% EtOH/2.5% Tween-80), imipramine (positive control; 60 mg/kg), or Test Compound 60 minutes before the start of the 5 minute test period. All injections are given using 1 cc tuberculin syringe with 26 ⅜ gauge needles (Becton-Dickinson, VWR Scientific, Bridgeport, N.J.). The volume of injection was 1 ml/kg.

Experimental Design

The procedure used in this study is similar to that previously described (Porsolt, et al., 1978), except the water depth is 31 cm in this procedure. The greater depth in this test prevents the rats from supporting themselves by touching the bottom of the cylinder with their feet. Swim sessions are conducted by placing rats in individual plexiglass cylinders (46 cm tall×20 cm in diameter) containing 23-25° C. water 31 cm deep. Swim tests are conducted always between 900 and 1700 hours and consist of an initial 15-min conditioning test followed 24 h later by a 5-minute test. Drug treatments are administered 60 minutes before the 5-minute test period. Following all swim sessions, rats are removed from the cylinders, dried with paper towels and placed in a heated cage for 15 minutes and returned to their home cages. All test sessions are videotaped using a color video camera and recorded for scoring later.

Behavioral Scoring

A rat's behavior is rated at 5-second intervals during the 5-minute test by a single individual, who is blind to the treatment condition. Scored behaviors are:
1. Immobility—rat remains floating in the water without struggling and is only making those movements necessary to keep its head above water;
2. Climbing—rat is making active movements with its forepaws in and out of the water, usually directed against the walls;
3. Swimming—rat is making active swimming motions, more than necessary to merely maintain its head above water, e.g. moving around in the cylinder; and
4. Diving—entire body of the rat is submerged.
   Data Analysis

The forced swim test data (immobility, swimming, climbing, diving) are subjected to a randomized, one-way ANOVA and post hoc tests conducted using the Newman-Keuls test. The data are analyzed using the GraphPad Prism (v2.01) (GraphPad Software, Inc., San Diego, Calif.).

2. Forced Swim Test (FST) in the Mouse

Animals

DBA/2 mice (Taconic Farms, N.Y.) are used in all experiments. Animals are housed 5 per cage in a controlled environment under a 12:12 hour light:dark cycle. Animals are handled 1 min each day for 4 days prior to the experiment. This procedure includes a mock gavage with a 1.5 inch feeding tube.

Drug Administration

Animals are randomly assigned to receive a single administration of vehicle (5% EtOH/5% Tween-80), Test Compound, or imipramine (60 mg/kg) by oral gavage 1 hour before the swim test.

Experimental Design

The procedure for the forced swim test in the mouse is similar to that described above for the rat, with some modifications. The cylinder used for the test is a 1-liter beaker (10.5cm diameter×15 cm height) fill to 800 ml (10 cm depth) of 23-25° C. water. Only one 5-minute swim test is conducted for each mouse, between 1300 and 1700 hours. Drug treatments are administered 30-60 minutes before the 5-minute test period. Following all swim sessions, mice are removed from the cylinders, dried with paper towels and placed in a heated cage for 15 minutes. All test sessions are videotaped using a Sony color video camera and recorder for scoring later.

Behavioral Scoring

The behavior during minutes 2-5 of the test is played back on a TV monitor and scored by the investigator. The total time spent immobile (animal floating with only minimal movements to remain afloat) and mobile (swimming and movements beyond those required to remain afloat) are recorded.

Data Analysis

The forced swim test data (time exhibiting immobility, mobility; seconds) are subjected to a randomized, one-way ANOVA and post hoc tests conducted using the Newman-Keuls test. The data are analyzed using the GraphPad Prism (v2.01) (GraphPad Software, Inc., San Diego, Calif.).

C. Anxiety

The following method describes a protocol that may be used to predict the efficacy of MCH1 antagonists for the treatment of anxiety.

Social Interaction Test (SIT)

Rats are allowed to acclimate to the animal care facility for 5 days and are housed singly for 5 days prior to testing. Animals are handled for 5 minutes per day. The design and procedure for the Social Interaction Test is carried out as previously described by Kennett, et al. (1997). On the test day, weight matched pairs of rats (±5%), unfamiliar to each other, are given identical treatments and returned to their home cages. Animals are randomly divided into 5 treatment groups, with 5 pairs per group, and are given one of the following i.p. treatments: Test Compound (10, 30 or 100 mg/kg), vehicle (1 ml/kg) or chlordiazepoxide (5 mg/kg). Dosing is 1 hour prior to testing. Rats are subsequently placed in a white perspex test box or arena (54×37×26 cm), whose floor is divided up into 24 equal squares, for 15 minutes. An air conditioner is used to generate background noise and to keep the room at approximately 74° F. All sessions are videotaped using a JVC camcorder (model GR-SZ1, Elmwood Park, N.J.) with either TDK (HG ultimate brand) or Sony 30 minute videocassettes. All sessions are conducted between 1300-1630 hours. Active social interaction, defined as grooming, sniffing, biting, boxing, wrestling, following and crawling over or under, is scored using a stopwatch (Sportsline model no. 226, 1/100 sec. discriminability). The number of episodes of rearing (animal completely raises up its body on its hind limbs), grooming (licking, biting, scratching of body), and face washing (i.e. hands are moved repeatedly over face), and number of squares crossed are scored. Passive social interaction (animals are lying beside or on top of each other) is not scored. All behaviors are assessed later by an observer who is blind as to the treatment of each pair. At the end of each test, the box is thoroughly wiped with moistened paper towels.

Animals

Male albino Sprague-Dawley rats (Taconic Farms, N.Y.) are housed in pairs under a 12 hr light dark cycle (lights on at 0700 hrs.) with free access to food and water.

Drug Administration

Test Compound is dissolved in either 100% DMSO or 5% lactic acid, v/v (Sigma Chemical Co., St. Louis, Mo.). Chlordiazepoxide (Sigma Chemical Co., St. Louis, Mo.) is dissolved in double distilled water. The vehicle consists of 50% DMSO (v/v) or 100% dimethylacetamide (DMA). All drug solutions are made up 10 minutes prior to injection and the solutions are discarded at the end of the test day. The volume of drug solution administered is 1 ml/kg.

Data Analysis

The social interaction data (time interacting, rearing and squares crossed) are subjected to a randomized, one-way ANOVA and post hoc tests conducted using the Student-Newman-Keuls test. The data are subjected to a test of normality (Shapiro-Wilk test). The data are analyzed using the GBSTAT program, version 6.5 (Dynamics Microsystems, Inc., Silver Spring, Md., 1997).

D. Urinary Disorders

The effects of compounds on the micturition reflex may be evaluated using the “distension-induced rhythmic contraction” (DIRC), as described in previous publications (e.g. Maggi et al, 1987;. Morikawa et al, 1992), and/or the Continuous Slow Transvesicular Infusion (CSTI) model.

1. DIRC Model

Female Sprague Dawley rats weighing approximately 300 g are anesthetized with subcutaneous urethane (1.2 g/kg). The trachea is cannulated with PE240 tubing to provide a clear airway throughout the experiment. A midline abdominal incision is made and the left and right ureters are isolated. The ureters are ligated distally (to prevent escape of fluids from the bladder) and cannulated proximally with PE10 tubing. The incision is closed using 4-0 silk sutures, leaving the PE10 lines routed to the exterior for the elimination of urine. The bladder is canulated via the transurethral route using PE50 tubing inserted 2.5 cm beyond the urethral opening. This cannula is secured to the tail using tape and connected to a pressure transducer. To prevent leakage from the bladder, the cannula is tied tightly to the exterior urethral opening using 4-0 silk. To initiate the micturition reflex, the bladder is first emptied by applying pressure to the lower abdomen, and then filled with normal saline in 100 increments (maximum=2 ml) until spontaneous bladder contractions occurred (typically 20-40 mmHg at a rate of one contraction every 2 to 3 minutes. Once a regular rhythm is established, vehicle (saline) or Test Compounds are administered i.v. or i.p. to explore their effects on bladder activity. The 5-HT_{1A} antagonist WAY-100635 is often given as a positive control. Data are expressed as contraction interval (in seconds) before drug application (basal), or after the application of vehicle or test article.

2. Continuous Slow Transvesicular Infusion (CSTI) Rat Model

Male Sprague Dawley rats weighing approximately 300 g are used for the study. Rats are anaesthetized with pentobarbitone sodium (50 mg/kg, i.p). Through a median abdominal incision, the bladder is exposed and a polyethylene cannula (PE 50) is introduced into the bladder through a small cut on the dome of the bladder and the cannula is secured with a purse string suture. The other end of the cannula is exteriorized subcutaneously at the dorsal neck area. Similarly, another cannula (PE 50) is introduced into the stomach through a paramedian abdominal incision with the free end exteriorized subcutaneously to the neck region. The surgical wounds are closed with silk 4-0 suture and the animal is allowed to recover with appropriate post surgical care. On the following day, the animal is placed in a rat restrainer. The open end of the bladder- cannula is connected to a pressure transducer as well as infusion pump through a three-way stopcock. The bladder voiding cycles are initiated by continuous infusion of normal saline at the rate of 100 μl/min. The repetitive voiding contractions are recorded on a Power Lab on-line data acquisition software. After recording the basal voiding pattern for an hour, the test drug or vehicle is administered directly into stomach through the intragastric catheter and the voiding cycles are monitored for 5 hours. Micturition pressure and frequency are calculated before and after the treatment (at every 30 min interval) for each animal. Bladder capacity is calculated from the micturition frequency, based on the constant infusion of 100 ul/min. The effect of the test drug is expressed as a percentage of basal, pre-drug bladder capacity. WAY 100635 is often used as positive control for comparison.

References

Bednarek, M. A., et al. “Synthesis and biological evaluation in vitro of a selective, high potency peptide agonist of human melanin-concentrating hormone action at human melanin-concentrating hormone receptor 1” J Biol Chem. 277(16): 13821-13826 (2002).
Borowsky, B., et al., Antidepressant, anxiolytic and anorectic effects of a melanin-concentrating hormone-1 receptor antagonist”. Nature Medicine, 8. (8): pg 825-830 (2002).
Chambers, J., et al., “Melanin-concentrating hormone is the cognate ligand for the orphan G-protein-coupled receptor SLC-1” Nature 400(6741): 261-6 (1999).
Chen, Y., et al, “Targeted disruption of the melanin-concentrating hormone receptor-1 results in hyperphagia and resistance to diet-induced obesity” Endocrinology 143(7): 2469-2477(2002).
Doggrell S A., “Does the melanin-concentrating hormone antagonist SNAP-7941 deserve 3As? Expert Opinion on Investigational Drugs; 2003, 121(6): pp. 1035-1038 (2003).
Kawauchi, H., et al., Characterization of melanin-concentrating hormone in chum salmon pituitaries. Nature, 305: 321-333 (1983).
Lakaye, B., et al., “Cloning of the rat brain cDNA encoding for the SLC-1 G protein-coupled receptor reveals the presence of an intron in the gene” Biochem Biophys Acta 1401(2): 216-220 (1998).
Maggi, C. A., et al., “Spinal and supraspinal components of GABAergic inhibition of the micturition reflex in rats.” J Pharmacol Exp Ther 240: 998-1005 (1987).
Marsh, D. J., et al, “Melanin-concentrating hormone 1 receptor-deficient mice are lean, hyperactive, and hyperphagic and have altered metabolism” Proc Natl Acad Sci U S A 99(5): 3240-3245 (2002).
Saito, Y., et al., “Molecular characterization of the melanin-concentrating-hormone receptor” Nature, 400(6741): 265-269 (1999).
Porsolt, R. D., et al., “Behavioural despair in rats: a new model sensitive to antidepressant treatments” Eur J Pharmacol 47(4): 379-391 (1978).
Takekawa, S., et al., “T-226296: a novel, orally active and selective melanin-concentrating hormone receptor antagonist” Eur J Pharmacol 438(3): 129-35 (2002).

1. A compound having the structure: 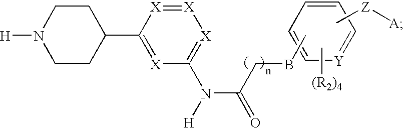
wherein each X is independently CR₁, or N, with the proviso that if one X is N then the remaining X are CR₁;
wherein each R₁ is independently —H, —F, —Cl, —Br, —I, —CN, —NO₂, straight chained or branched C₁-C₇ alkyl, alkyloxy, monofluoroalkyl or polyfluoroalkyl, or C₃-C₆ cycloalkyl —C₁-C₇ alkyl;
wherein each R₂ is independently —H, —F, —Cl, —Br, —I, —CN, —NO₂ or straight chained or branched C₁-C₇ alkyl, monofluoroalkyl or polyfluoroalkyl;
wherein n is an integer from 2 to 6 inclusive;
wherein B is CH₂, CHOH, O or CO;
wherein Y is C or N;
wherein Z is O, S, SO, SO₂, CH₂, CO, CHOH or null;
and wherein A is phenyl or heteroaryl, where the phenyl or heteroaryl is optionally substituted with three or less R₂;
or a pharmaceutically acceptable salt thereof.

2. The compound of claim 1, wherein n is 2 or 3.

3. The compound of claim 2, wherein each R₁ is independently —H, —F, —Cl, straight chained or branched C₁-C₄ alkyl or alkoxy, or C₃-C₆ cycloalkyl-C₁-C₄ alkyl.

4. The compound of claim 3, wherein each R₂ is independently —H, —F, —Cl, or straight chained or branched C₁-C₄ alkyl, monofluoroalkyl or polyfluoroalkyl.

5. The compound of claim 4, wherein one X is N.

6. The compound of claim 4, wherein each X is CR₁.

7. The compound of claim 6, wherein A is pyridinyl optionally substituted with three or less R₂.

8. The compound of claim 6, wherein A is thienyl optionally substituted with three or less R₂.

9. The compound of claim 6, wherein A is furanyl optionally substituted with three or less R₂.

10. The compound of claim 6, wherein A is thiazolyl optionally substituted with three or less R₂.

11. The compound of claim 6, wherein A is imidazolyl optionally substituted with three or less R₂.

12. The compound of claim 6, wherein A is pyrazolyl optionally substituted with three or less R₂.

13. The compound of claim 6, wherein A is oxazolyl optionally substituted with three or less R₂.

14. The compound of claim 6, wherein A is triazinyl optionally substituted with three or less R₂.

15. The compound of claim 6, wherein A is phenyl optionally substituted with three or less R₂.

16. The compound of claim 15, wherein Z is S, SO or SO₂.

17. The compound of claim 15, wherein Z is CH₂, CO or CHOH.

18. The compound of claim 15, wherein Z is O or null.

19. The compound of claim 18, wherein B is CH₂.

20. The compound of claim 19, wherein Z is O.

21. The compound of claim 20, wherein the compound is selected from the group consisting of 5-(2-Phenoxyphenyl)-N-(3-(4-piperidyl)phenyl)pentanamide; N-(4-Methyl-3-(4-piperidyl) phenyl)-5-(2-phenoxyphenyl)pentanamide; N-(4-Fluoro-3-(4-piperidyl)phenyl)-5-(2-phenoxy phenyl)pentanamide; N-(2-Fluoro-5-(4-piperidyl)phenyl)-5-(2-phenoxyphenyl)pentanamide; and N-(2-Fluoro-4-methyl-5-(4-piperidyl)phenyl)-5-(2-phenoxyphenyl)pentanamide.

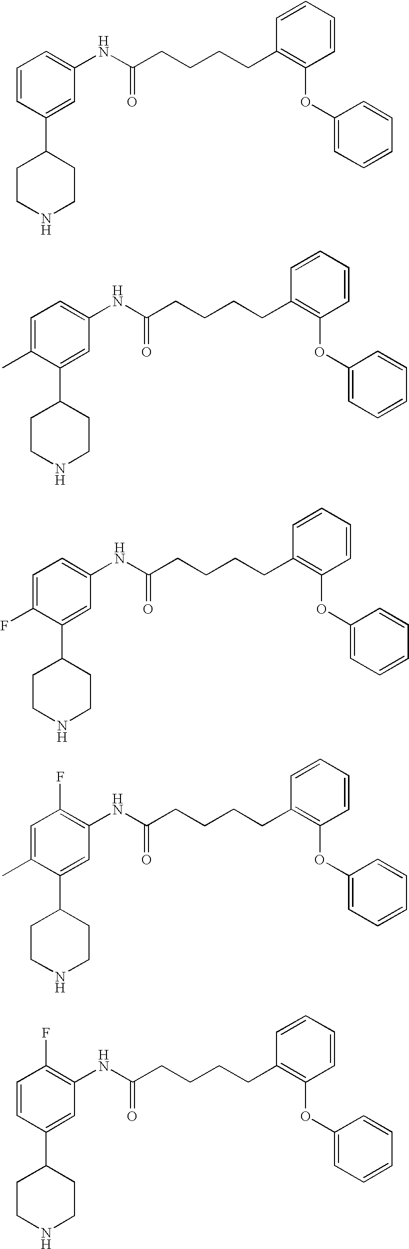

22. The compound of claim 19, wherein Z is null.

23. The compound of claim 22, wherein the compound is selected from the group consisting of N-(4-Methyl-3-(4-piperidyl)phenyl)-5-(4-phenylphenyl)pentanamide; N-(2-Fluoro-5-(4-piperidyl)phenyl)-5-(4-phenylphenyl)pentanamide; N-(2-Fluoro-4-methyl-5-(4-piperidyl)phenyl)-5-(4-phenylphenyl)pentanamide; and N-(4-fluoro-3-(4-piperidyl)phenyl)-5-(4-phenylphenyl)pentanamide.

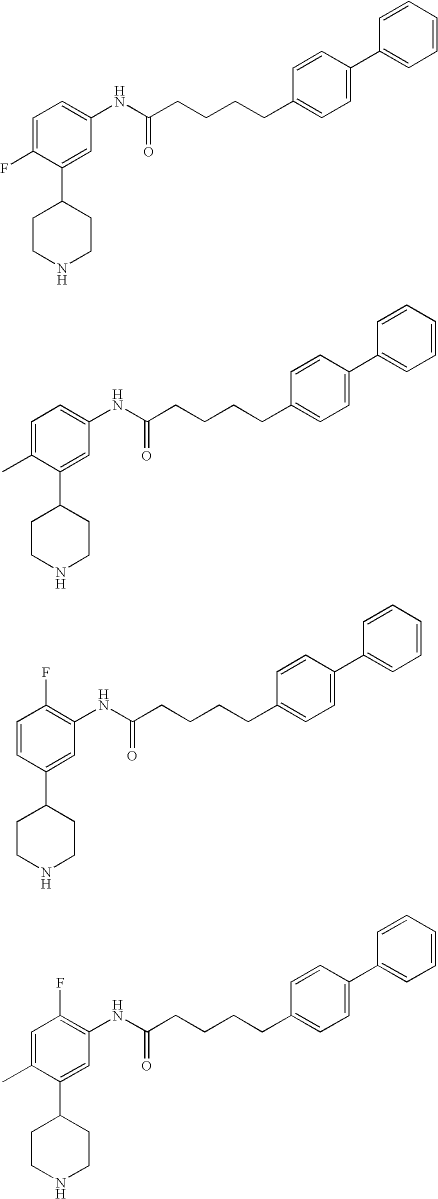

24. The compound of claim 18, wherein B is CO.

25. The compound of claim 24, wherein Z is null.

26. The compound of claim 25, wherein the compound is selected from the group consisting of N-(4-fluoro-3-(4-piperidyl)phenyl)-4-oxo-4-(4-phenylphenyl)butanamide; N-(2-fluoro-5-(4-piperidyl)phenyl)-4-oxo-4-(4-phenylphenyl) butanamide; N-(4-fluoro-3-(4-piperidyl)phenyl)-5-oxo-5-(4-phenylphenyl)pentanamide; 5-oxo-5-(4-phenylphenyl)-N-(3-(4-piperidyl)phenyl) pentanamide; N-(4-methyl-3-(4-piperidyl)phenyl)-5-oxo-5-(4-phenylphenyl)pentanamide; N-(2-fluoro-5-(4-piperidyl)phenyl)-5-oxo-5-(4-phenylphenyl)pentanamide; and N-(2,4-difluoro-5-(4-piperidyl)phenyl)-4-oxo-4-(4-phenylphenyl)butanamide.

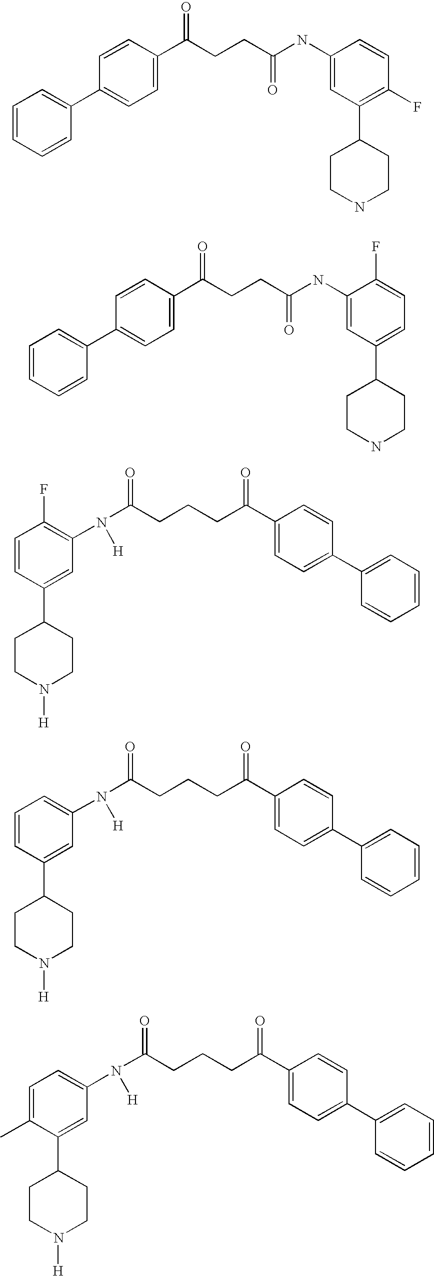

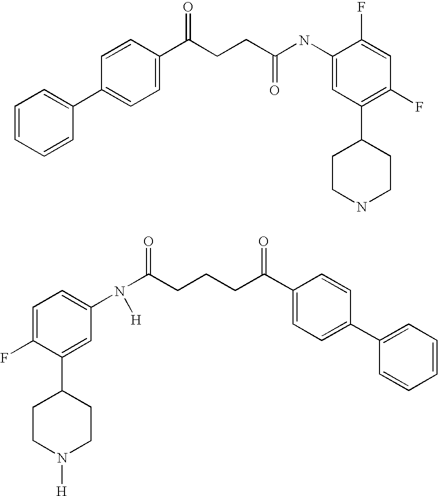

27. The compound of claim 1, wherein the compound is enantiomerically pure.

28. The compound of claim 1, wherein the compound is diastereomerically pure.

29. A pharmaceutical composition that comprises a therapeutically effective amount of the compound of claim 1 and a pharmaceutically acceptable carrier.

30. A pharmaceutical composition made by admixing a compound of claim 1 and a pharmaceutically acceptable carrier.

31. A process for making a pharmaceutical composition comprising admixing a compound of claim 1 and a pharmaceutically acceptable carrier.

32. A method of treating a subject suffering from an affective disorder selected from the group consisting of depression, major depression, bipolar disorder, agoraphobia, specific phobia, social phobia, obsessive-compulsive disorder, post-traumatic stress disorder, acute stress disorder and anxiety, comprising administering to the subject a therapeutically effective amount of the compound of claim 1.

33. A method of treating a subject suffering from a urinary disorder selected from the group consisting of urinary incontinence, urge incontinence, urinary frequency, urinary urgency, nocturia and enuresis comprising administering to the subject a therapeutically effective amount of the compound of claim 1.

34. A method of treating a subject suffering from an eating disorder selected from the group consisting of obesity, bulimia, bulimia nervosa and anorexia nervosa comprising administering to the subject a therapeutically effective amount of the compound of claim 1.
